(19) United States
(12) Patent Application Publication
(10) Pub. No.: US 2026/0131006 A1
(43) Pub. Date: May 14, 2026

(54) PHARMACEUTICAL COMPOSITIONS USING NON-ANIMAL ORIGIN MATRIX FORMERS AND METHODS OF MAKING THEREOF

(71) Applicant: Catalent U.K. Swindon Zydis Limited, Glasgow (GB)

(72) Inventors: Malgorzata KLETA, Swindon (GB); Vikram CHOUK, Swindon (GB)

(73) Assignee: Catalent U.K. Swindon Zydis Limited, Glasgow (GB)

(21) Appl. No.: 19/388,478

(22) Filed: Nov. 13, 2025

Related U.S. Application Data
(60) Provisional application No. 63/720,343, filed on Nov. 14, 2024.

(51) Int. Cl. A61K 47/38; A61K 9/16; A61K 9/19; A61K 31/192; A61K 31/198; A61K 31/4545; A61K 47/26; A61K 47/36

(52) U.S. Cl. CPC A61K 47/38; A61K 9/1694; A61K 9/19; A61K 31/192; A61K 31/198; A61K 31/4545; A61K 47/26; A61K 47/36

(57) ABSTRACT

The present disclosure is directed to pharmaceutical compositions using non-animal-based matrix formers and method of making such pharmaceutical compositions. The pharmaceutical compositions can include an active pharmaceutical ingredient, a non-gelatin matrix former, and a structure former. Such a composition can offer comparable and/or improved characteristics compared to other gelatin pharmaceutical compositions.

### CROSS-REFERENCE TO RELATED APPLICATIONS

This application claims the benefit of U.S. Provisional Application No. 63/720,343 filed Nov. 14, 2024, the entire contents of which are incorporated herein by reference.

### FIELD OF THE DISCLOSURE

This disclosure relates generally to pharmaceutical compositions comprising non-animal origin matrix formers, and methods for making thereof. Specifically, this disclosure relates to pharmaceutical compositions comprising non-gelatin matrix formers, and methods for making thereof.

### BACKGROUND OF THE DISCLOSURE

Tablets are the most common pharmaceutical dosage form. However, many patients have poor compliance due to difficulty in swallowing the tablet. Further, ineffective dissolution of conventional tablets can cause a lack of bioavailability of the active pharmaceutical ingredients (“APIs”). Orally disintegrating/dissolvable tablets (“ODTs”) have been proposed to overcome the shortcomings of conventional tablets. Many ODT pharmaceutical compositions, in particular those in the lyophilized form, are known to be sensitive to environmental conditions such as humidity and temperature. Further, creation of such a lyophilized ODT may require use of an animal-based matrix former, such as a gelatin matrix former. Use of a gelatin matrix former may not be suitable for patients with moral, religious, allergic, and dietary restrictions or aversions to gelatin products. The gelatin allergy can cause a severe reaction, including anaphylaxis. Further, many mammalian-derived gelatin products are classified as TSE-risk materials and require Transmissible Spongiform Encephalopathies (“TSE”)/Bovine Spongiform Encephalopathy (“BSE”) compliance certificates issued by country-specific authorities. Additionally, gelatin-based compositions are prone to bacterial growth which must be controlled by strict in-process microbiological quality control.

### SUMMARY OF THE DISCLOSURE

Gelatin, though undesirable at least for some of the reasons described above, does provide many desirable characteristics when used as a matrix former in lyophilized ODTs. A lyophilized ODT that replaces gelatin as a matrix former with a non-animal-based and/or non-gelatin matrix former should perform at least as well as a gelatin matrix former, particularly in terms of disintegration time, morphology, and/or a low water content. The low water content can enable better stability in humid environments, resistance to polymorphic/pseudo-polymorphic changes of a drug, and/or ensure that no stringent hermetic packaging may be needed.

Applicant has discovered pharmaceutical compositions using non-animal-based or non-animal origin (e.g., non-gelatin) matrix formers and methods for making such. Specifically, Applicant has discovered a pharmaceutical composition that can deliver an API using a non-gelatin matrix former that can retain many of the desirable properties of compositions that use animal-based (e.g., gelatin) matrix formers. Use of non-gelatin matrix formers may also improve tablet taste. Additionally, using non-animal origin matrix formers can reduce risk of microbial growth compared to animal-based matrix formers. There may be no need to control for TSEs and BSEs and/or comply with the legal requirements/procedures associated with each due to the absence of the animal origin matrix formers.

As previously mentioned, it may be desirable to create compositions that use non-gelatin matrix formers that perform at least as well as compositions that use gelatin matrix formers. For example, gelatin matrix formers in lyophilized ODTs provide desirable dissolution characteristics. Applicant discovered that using these non-animal-based matrix formers, either alone or in combination, can make it possible to produce pharmaceutical compositions for use in ODTs with drug dissolution profiles equivalent to those with gelatin. In some embodiments, use of these non-gelatin matrix formers may also not affect the disintegration characteristic requirements of the lyophilized ODTs.

Using gelatin matrix formers may also result in low water content in the lyophilized ODTs. However, Applicant discovered through analytical studies that use of these non-gelatin matrix formers can lead to lyophilized ODTs with lower water content when lyophilized when compared to those using gelatin matrix formers. This can result in beneficial product stability when stored, particularly when exposed to high-humidity environments. This may also allow the use of less stringent moisture protective packaging required for the lyophilized ODTs. In addition, some APIs are sensitive to moisture, and exposure to moisture may lead to polymorphic transformation or hydrolysis upon storage. A lower residue water content in the lyophilized ODT can ensure greater stability for these moisture-sensitive APIs.

In some embodiments, a pharmaceutical composition includes an active pharmaceutical ingredient (API), 1-45 wt. % non-gelatin matrix former, and 1-45 wt. % structure former. In some embodiments, the non-gelatin matrix former comprises hydroxypropyl methylcellulose, methyl cellulose, pullulan, poly vinyl alcohol, or combinations thereof. In some embodiments, the pharmaceutical composition comprises 1-75 wt. % API. In some embodiments, the structure former comprises sugar, isomalt, mannitol, dextrose, lactose, galactose, cyclodextrin, or combinations thereof. In some embodiments, the structure former comprises isomalt. In some embodiments, the pharmaceutical composition further comprises a pH modifier. In some embodiments, the pharmaceutical composition comprises 0.001-2 wt. % pH modifier. In some embodiments, the pH modifier comprises citric acid. In some embodiments, the pharmaceutical composition further comprises a flavoring agent. In some embodiments, the pharmaceutical composition comprises 0.001-1 wt. % flavoring agent. In some embodiments, the flavoring agent comprises flavor mint. In some embodiments, the pharmaceutical composition is configured to dissolve in less than 30 seconds. In some embodiments, the pharmaceutical composition comprises at most 4.5 wt. % water. In some embodiments, the pharmaceutical composition is an orally dissolvable tablet.

In some embodiments, a method of making a pharmaceutical composition, the method comprising: dosing a pharmaceutical formulation into a preformed mold, the pharmaceutical formulation comprising: a pharmaceutical active ingredient; 1-20 wt. % non-gelatin matrix former; and 1-20 wt, % structure former; freezing the dosed pharmaceutical formulation; and freeze-drying the frozen pharmaceutical formulation to form the pharmaceutical composition. In some embodiments, the dosed pharmaceutical formulation is frozen at a temperature of −40° C. to −120° C. for a duration of about 1-5 minutes. In some embodiments, the non-gelatin matrix former comprises hydroxypropyl methylcellulose, methyl cellulose, pullulan, poly vinyl alcohol, or combinations thereof. In some embodiments, the pharmaceutical composition comprises 1-45 wt. % API. In some embodiments, the API comprises an antihistamine. In some embodiments, the antihistamine comprises loratadine. In some embodiments, the structure former is a sugar or sugar-like structure former. In some embodiments, the structure former comprises isomalt. In some embodiments, the pharmaceutical composition further comprises a pH modifier. In some embodiments, the pharmaceutical composition comprises 0.001-5 wt. % pH modifier. In some embodiments, the pH modifier comprises citric acid. In some embodiments, the pharmaceutical composition further comprises a flavoring agent. In some embodiments, the pharmaceutical composition comprises 0.001-5 wt. % flavoring agent. In some embodiments, the flavoring agent comprises flavor mint. In some embodiments, the pharmaceutical composition is configured to dissolve in less than 30 seconds. In some embodiments, the pharmaceutical composition comprises at most 4.5 wt. % water. In some embodiments, the pharmaceutical composition is an orally dissolvable tablet.

In some embodiments, a pharmaceutical composition includes an active pharmaceutical ingredient (“API”); 1-45 wt. % non-gelatin matrix former comprising hydroxypropyl methylcellulose, methyl cellulose, pullulan, or combinations thereof; and 1-45 wt. % structure former. In some embodiments, the pharmaceutical composition comprises 1-95 wt. % API. In some embodiments, the structure former comprises sugars, polyols, or combinations thereof. In some embodiments, the structure former comprises isomalt, sorbitol, mannitol, sucralose, trehalose, dextrose, lactose, galactose, cyclodextrin, or combinations thereof. In some embodiments, the structure former comprises isomalt, sorbitol, mannitol, sucralose, trehalose, or combinations thereof. In some embodiments, the pharmaceutical composition further comprises a pH modifier. In some embodiments, the pharmaceutical composition comprises 0.001-2 wt. % pH modifier. In some embodiments, the pH modifier comprises citric acid or sodium hydroxide. In some embodiments, the pharmaceutical composition further comprises a flavoring agent. In some embodiments, the pharmaceutical composition comprises 0.001-1 wt. % flavoring agent. In some embodiments, a disintegration time of the pharmaceutical composition is less than 7 seconds. In some embodiments, the pharmaceutical composition comprises at most 5 wt. % water. In some embodiments, the pharmaceutical composition comprises at most 4.5 wt. % water. In some embodiments, the pharmaceutical composition is an orally dissolvable tablet. In some embodiments, a percent dissolution of the pharmaceutical composition and corresponding API after 6 minutes is at least 75% of label claim. In some embodiments, a water activity of the pharmaceutical composition is 0.2-0.45. In some embodiments, the non-gelatin matrix former exhibits a narrow hysteresis loop during dynamic vapor sorption analysis, indicating moderate porosity and reversible moisture uptake. In some embodiments, the API is embedded in the non-gelatin matrix former such that the non-gelatin matrix former provides reversible moisture sorption characteristics and reduced bound water retention compared to a gelatin matrix former. In some embodiments, the pharmaceutical composition maintains physical integrity and stability under fluctuating humidity conditions due to the reversible moisture sorption behavior of the non-gelatin matrix former. In some embodiments, the non-gelatin matrix former exhibits selective buffering behavior with limited buffering capacity between a pH of 4-10. In some embodiments, the non-gelatin matrix former resists rapid pH changes upon addition of acidic or alkaline agents outside the pH range of 4-10.

In some embodiments, a method of making a pharmaceutical composition includes dosing a pharmaceutical formulation into a preformed mold, the pharmaceutical formulation comprising: an active pharmaceutical ingredient (“API”); 1-20 wt. % non-gelatin matrix former comprising hydroxypropyl methylcellulose, methyl cellulose, pullulan, or combinations thereof; and 1-20 wt, % structure former; freezing the dosed pharmaceutical formulation; and freeze-drying the frozen pharmaceutical formulation to form the pharmaceutical composition. In some embodiments, the dosed pharmaceutical formulation is frozen at a temperature of −40° C. to −120° C. for a duration of about 1-5 minutes. In some embodiments, the pharmaceutical formulation comprises 1-45 wt. % API. In some embodiments, the structure former comprises sugars, polyols, or combinations thereof. In some embodiments, the structure former comprises isomalt, mannitol, sorbitol, sucralose, trehalose, dextrose, lactose, galactose, cyclodextrin, or combinations thereof. In some embodiments, the structure former comprises isomalt, mannitol, or combinations thereof. In some embodiments, the pharmaceutical formulation further comprises a pH modifier. In some embodiments, the pharmaceutical formulation comprises 0.001-5 wt. % pH modifier. In some embodiments, the pH modifier comprises citric acid or sodium hydroxide. In some embodiments, the pharmaceutical formulation further comprises a flavoring agent. In some embodiments, the pharmaceutical formulation comprises 0.001-5 wt. % flavoring agent.

In some embodiments, a pharmaceutical composition includes: 25-35 wt. % an active pharmaceutical ingredient (“API”); 20-45 wt. % non-gelatin matrix former comprising hydroxypropyl methylcellulose, methyl cellulose, pullulan, or combinations thereof; and 20-45 wt. % structure former comprising isomalt, mannitol, sorbitol, sucralose, trehalose, or combinations thereof. In some embodiments, the API is loratadine.

In some embodiments, a pharmaceutical composition includes: 40-60 wt. % an active pharmaceutical ingredient (“API”); 20-35 wt. % non-gelatin matrix former comprising hydroxypropyl methylcellulose, methyl cellulose, pullulan, or combinations thereof; and 15-35 wt. % structure former comprising isomalt, mannitol, sorbitol, sucralose, trehalose or combinations thereof. In some embodiments, the API is ibuprofen lysine.

In some embodiments, a pharmaceutical composition includes: 85-95 wt. % an active pharmaceutical ingredient (“API”); 1-5 wt. % non-gelatin matrix former comprising hydroxypropyl methylcellulose, methyl cellulose, pullulan, or combinations thereof; and 1-5 wt. % structure former comprising isomalt, mannitol, sorbitol, sucralose, trehalose, or combinations thereof. In some embodiments, the API is ibuprofen.

Additional advantages will be readily apparent to those skilled in the art from the following detailed description. The examples and descriptions herein are to be regarded as illustrative in nature and not restrictive.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE FIGURES

Exemplary embodiments are described with reference to the accompanying figures, in which:

FIG. 1 illustrates an exemplary flow chart for making a pharmaceutical composition in accordance with some embodiments disclosed herein.

FIG. 2A illustrates lyophilized pharmaceutical compositions using hydroxypropyl methylcellulose (“HPMC”) as a matrix former in accordance with some embodiments disclosed herein.

FIG. 2B illustrates a cross-section of a lyophilized pharmaceutical composition using hydroxypropyl methylcellulose as a matrix former in accordance with some embodiments disclosed herein.

FIG. 3A illustrates lyophilized pharmaceutical compositions using methyl cellulose (“MC”) as a matrix former in accordance with some embodiments disclosed herein.

FIG. 3B illustrates a cross-section of a lyophilized pharmaceutical composition using methyl cellulose as a matrix former in accordance with some embodiments disclosed herein.

FIG. 4A illustrates lyophilized pharmaceutical compositions using Pullulan as a matrix former in accordance with some embodiments disclosed herein.

FIG. 4B illustrates a cross-section of a lyophilized pharmaceutical composition using Pullulan as a matrix former in accordance with some embodiments disclosed herein.

FIG. 5 is a table showing the results on appearance from the analytical studies disclosed herein.

FIG. 6 illustrates the results of the dissolution studies with MC as a matrix former at the 12 month time point in the analytical studies disclosed herein.

FIG. 7 illustrates the results of the dissolution studies with Pullulan as a matrix former done with the 3, 6, and 12 month time point in the analytical studies disclosed herein.

FIG. 8 illustrates the results of the dissolution studies with HPMC as a matrix former done with the 3, 6, and 12 month time point in the analytical studies disclosed herein.

FIG. 9A illustrates a graphical representation of the water content studies with HPMC as a matrix former in the analytical studies disclosed herein.

FIG. 9B illustrates a graphical representation of the water content studies with MC as a matrix former in the analytical studies disclosed herein.

FIG. 9C illustrates a graphical representation of the water content studies with Pullulan as a matrix former in the analytical studies disclosed herein.

FIG. 10A illustrates lyophilized pharmaceutical compositions using HPMC, MC, Pullulan, and Gelatin as a matrix former in accordance with some embodiments disclosed herein.

FIG. 10B illustrates a cross-section of a lyophilized pharmaceutical composition using HPMC and MC as a matrix former without an API (i.e., a placebo) in accordance with some embodiments disclosed herein.

FIG. 10C illustrates a cross-section of a lyophilized pharmaceutical composition using Pullulan and Gelatin as a matrix former without an API (i.e., a placebo) in accordance with some embodiments disclosed herein.

FIG. 11A illustrates the dynamic vapour sorption (DVS) results with HPMC as a matrix former in the analytical studies disclosed herein.

FIG. 11B illustrates the dynamic vapour sorption (DVS) results with Gelatin as a matrix former in the analytical studies disclosed herein.

FIG. 11C illustrates the dynamic vapour sorption (DVS) results with MC as a matrix former in the analytical studies disclosed herein.

FIG. 11D illustrates the dynamic vapour sorption (DVS) results with Pullulan as a matrix former in the analytical studies disclosed herein.

FIG. 11E illustrates the hysteresis results with HPMC and Gelatin as matrix formers in the analytical studies disclosed herein.

FIG. 11F illustrates the hysteresis results with MC and Pullulan as matrix formers in the analytical studies disclosed herein.

FIG. 12 illustrates buffering capacity results in the analytical studies disclosed herein.

### DETAILED DESCRIPTION OF THE DISCLOSURE

Disclosed herein are pharmaceutical compositions, formulations, and methods of making such using non-animal-based matrix formers. Specifically, the present disclosure relates to freeze dried oral dispersible or disintegrating pharmaceutical compositions that use non-animal-origin matrix formers that compare favorably to pharmaceutical compositions that use gelatin matrix formers.

As stated above, Applicant was able to develop a non-gelatin pharmaceutical composition that may be used in place of gelatin pharmaceutical compositions. Specifically, Applicant determined that use of non-animal-origin matrix formers, such as HPMC, MC, PVA, and Pullulan, can create pharmaceutical compositions with comparable or better characteristics compared to gelatin ones. Such compositions can have lower water content, meaning better stability in humid environments, better resistance to polymorphic/pseudo-polymorphic changes of a drug (particularly water-sensitive APIs), and/or lack of a need for stringent hermetic packaging. Such compositions can also be suitable for patients with moral, religious, gelatin allergic, and/or dietary commitments, and lack of gelatin means they do not need to control for TSEs and BSEs and go through the legal requirements associated with each. Such compositions can have immediate release profiles without affecting the disintegration time when compared with gelatin compositions (e.g., disintegrating in less than 10 seconds, as may be desirable for ODTs). Further, use of a mucoadhesive matrix can improve drug absorption and therefore bioavailability. These non-gelatin matrix formers may also improve tablet taste.

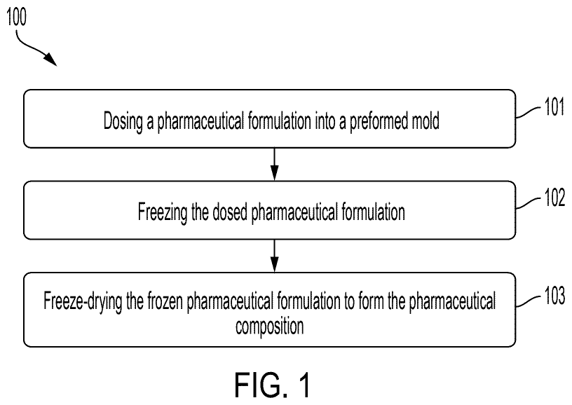

FIG. 1 illustrates a flow chart for method 100 of making pharmaceutical composition. The pharmaceutical composition created may be a lyophilized ODT.

At step 101, the pharmaceutical formulation may be dosed into a preformed mold. As used herein, “dosed” (or similar technology) refers to the deposition of a pre-determined aliquot of solution or suspension. As used herein, “preformed mold” refers to any suitable container or compartment into which an aqueous solution or suspension may be deposited and within which subsequently freeze dried. In some embodiments, the preformed mold is a blister pack with one or more blister pockets. Predetermined aliquots in an amount of about 50-2000 mg wet filling dosing weight of the pharmaceutical formulation can be metered into preformed molds. The minimum unit size (e.g., wet fill weight, 50 mg) can be selected to minimize the amount of API in solution proportionally to the unit dose, and therefore its surface area and potential for oxidative degradation in the final dosage form.

In some embodiments, the pharmaceutical formulation may comprise an active pharmaceutical ingredient (“API”), a non-animal-based (e.g., non-gelatin) matrix former, and/or a structure former.

Matrix formers can provide the network structure of the pharmaceutical composition that imparts strength and reliance during handling. In some embodiments, the matrix former can provide a framework and/or scaffold that can trap the active pharmaceutical ingredient (and/or other excipients) during the freeze-drying process. In some embodiments, the matrix former can form a continuous network that holds components of the freeze-dried dosage form (i.e., pharmaceutical composition) together, thereby providing rapid dissolution and/or uniform drug distribution. Many use gelatins (e.g., fish gelatin, bovine gelatin, porcine gelatin, or combinations thereof) as a matrix former. However, in some embodiments, the non-gelatin matrix former is non-animal-based/non-animal origin. This can make it suitable for patients with moral, dietary, and/or religious commitments that prevent them from consuming and/or using gelatin, gelatin, animal-based, or animal-origin products. In some embodiments, the matrix former may employ synthesized materials, hence the formulation can be suitable for many patients. Additionally, synthesized materials may reduce variability depending on environmental factors compared to gelatin products. Variables such as age, breed, climate (temperature, humidity), etc. can affect the manufacturing process. For example, when gelatin is submitted to pH and temperature variations, the material structure is known to change. Further, many mammalian-derived gelatin products are classified as TSE-risk materials and require TSE/BSE compliance certificates issued by country-specific authorities. Additionally, gelatin-based formulations are prone to bacterial growth, which must be controlled by strict in-process microbiological quality control.

In some embodiments, the pharmaceutical formulation includes about 1-20 wt. %, about 1-10 wt. %, about 2-8 wt. %, about 2-6 wt. %, about 2.5-5 wt. %, about 3-5 wt. %, about 3.5-4.5 wt. %, or about 2.5-4.5 wt. % non-gelatin matrix former. In some embodiments, the pharmaceutical formulation can include at least about 1 wt. %, at least about 1.5 wt. %, at least about 2 wt. %, at least about 2.5 wt. %, at least about 3 wt. %, at least about 4 wt. %, or at least about 5 wt. % non-gelatin matrix former. In some embodiments, the pharmaceutical formulation can include at most about 20 wt. %, at most about 10 wt. %, at most about 8 wt. %, at most about 6 wt. %, at most about 5 wt. %, at most about 4 wt. %, at most about 3 wt. %, at most about 2.5 wt. %, or at most about 2 wt. % non-gelatin matrix former.

In some embodiments, the non-gelatin matrix former is hydroxypropyl methylcellulose (“HPMC”). In some embodiments, the non-gelatin matrix former is methyl cellulose (“MC”). In some embodiments, the non-gelatin matrix former is Pullulan. In some embodiments, the non-gelatin matrix former is HPMC, MC, Pullulan, poly vinyl alcohol (“PVA”), or a combination thereof. In some embodiments, the non-gelatin matrix former is synthetic. These matrix formers may lead to improved taste for the pharmaceutical compositions.

In some embodiments, the pharmaceutical formulation may include a structure former. In some embodiments, the structure former can reinforce and/or stabilize the freeze-dried dosage form, thereby providing sufficient mechanical strength for handling and/or packaging. In some embodiments, the structure former can provide rigidity and prevent collapse to the freeze-dried dosage form. In some embodiments, the pharmaceutical formulation includes about 1-20 wt. %, about 1-10% wt., about 1-7 wt. %, about 2-5 wt. %, about 3-5 wt. %, about 2-4 wt. %, about 3-7.5 wt. %, about 3-6 wt. %, or about 4-6 wt. % structure former. In some embodiments, the pharmaceutical formulation can include at least about 1 wt. %, at least about 1.5 wt. %, at least about 2 wt. %, at least about 3 wt. %, at least about 4 wt. %, or at least about 5 wt. % structure former. In some embodiments, the pharmaceutical formulation can include at most about 20 wt. %, at most about 10 wt. %, at most about 7 wt. %, at most about 6 wt. %, at most about 5 wt. %, at most about 4 wt. %, at most about 3 wt. %, at most about 2.5 wt. %, or at most about 2 wt. % structure former.

In some embodiments, the structure former is a sugar, polyol, sugar-like structure former (e.g., sugar substitute), or combination thereof. Suitable structure formers can include, but are not limited to, sugars, polyols, isomalt, mannitol, sorbitol, sucralose, trehalose, dextrose, lactose, galactose, cyclodextrin, or combinations thereof. In some embodiments, the structure former includes isomalt, mannitol, or combinations thereof. In some embodiments, the structure former can be used in freeze drying as a building agent as it crystallizes to provide structural robustness to the freeze-dried product. In some embodiments, the structure former is isomalt, a combination of sorbitol and mannitol, mannitol, or combinations thereof. In some embodiments, the structure former includes GalenIQ™.

In some embodiments, the pharmaceutical formulation may include an API. In some embodiments, the pharmaceutical formulation can include about 1-60 wt. %, about 1-45 wt. %, about 1-30 wt. %, about 1-25 wt. %, about 1-20 wt. %, about 1-15 wt. %, 3-10 wt. %, or about 4 wt. % API (e.g., solid (crystalline) Loratadine). In some embodiments, the pharmaceutical formulation includes at least 1 wt. %, at least 2 wt. %, at least 3 wt. %, at least 4 wt. %, at least 5 wt. %, at least about 8 wt. %, at least about 10 wt. %, at least 15 wt. %, at least 20 wt. %, at least about 25 wt. %, at least about 30 wt. %, at least about 35 wt. %, or at least about 40 wt. % API. In some embodiments, the pharmaceutical formulation includes at most about 75 wt. %, at most about 70 wt. %, at most about 65 wt. %, at most about 60 wt. %, at most about 55 wt. %, at most about 50 wt. %, at most about 45 wt. %, at most about 40 wt. %, at most about 30 wt. %, at most about 25 wt. %, at most about 20 wt. %, at most about 15 wt. %, at most about 12 wt. %, at most about 10 wt. %, at most about 8 wt. %, at most about 6 wt. %, at most about 5 wt. %, or at most about 4 wt. % API.

As used herein, “active pharmaceutical ingredient” or “API” refers to a drug product that may be used in the diagnosis, cure, mitigation, treatment, or prevention of disease. Any API may be used for purposes of the present disclosure. In some embodiments, the APIs used herein can be any Biopharmaceutics Classification System (BCS) APIs. In some embodiments, the APIs used herein can be any API from the Biopharmaceutics Drug Disposition Classification System (BDDCS) disclosed in the research article titled “BDDCS Applied to Over 900 Drugs” from The AAPS Journal, Vol. 13, No. 4, December 2011, which is hereby incorporated by reference in its entirety.

In some embodiments, the API can be a moisture sensitive API. In some embodiments, the non-gelatin matrix former can enhance the pharmaceutical composition stability with the moisture-sensitive API because the non-gelatin matrix former can provide reversible moisture sorption characteristics and reduced bound water retention compared to gelatin matrices. In some embodiments, the pharmaceutical compositions can maintain physical integrity and stability under fluctuating humidity conditions due to the reversible moisture sorption behavior of the non-gelatin matrix former.

Suitable APIs include, without limitation: analgesics and anti-inflammatory agents (e.g., nonsteroidal anti-inflammatory drug), antacids, anthelmintics, anti-arrhythnic agents, anti-bacterial agents, anti-coagulants, anti-depressants, anti-diabetics, anti-diarrheals, anti-epileptics, anti-fungal agents, anti-gout agents, antihypertensive agents, anti-malarials, anti-migraine agents, anti-muscarinic agents, anti-neoplastic agents and immunosuppressants, anti-protazoal agents, anti-psychotics, anti-emetics, antirheumatics, anti-thyroid agents, antivirals, anxiolytics, aperients, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, laxatives, lipid regulating agents, local anesthetics, neuromuscular agents, nitrates and anti-anginal agents, nutritional agents, opioid analgesics, oral vaccines, proteins, peptides and recombinant drugs, purgatives, sex hormones and contraceptives, spermicides, and stimulants; and combinations thereof. In some embodiments, the API is an anti-histamine (e.g., Loratadine). In some embodiments, the API can be an anti-inflammatory agent such as Ibuprofen or Ibuprofen lysine. Lists of specific examples of these API may be found in U.S. Pat. No. 6,709,669 and U.S. Pat. Pub. 2024/0374524, both of which are incorporated herein by reference in their entirety. When present, the API can be present in the pharmaceutical formulation or pharmaceutical composition (e.g., lyophilized ODT) in an amount that is necessary to exhibit the required physiological effect as established by clinical studies One of ordinary skill in the art can readily determine an appropriate amount of API to include in the dosage form made according to the present disclosure.

In some embodiments, the API is included in the pharmaceutical formulation disclosed herein in an amount, which is sufficient to render it pharmaceutically effective when provided in a pharmaceutical composition (e.g., lyophilized ODT). A person of skill in the art can readily determine the pharmaceutically effective amount for a given disease or infection based on, among other facts, age and weight of the patient to whom the pharmaceutical composition will be administered.

The pharmaceutical formulation may also contain additional pharmaceutically acceptable agents or excipients. Such additional pharmaceutically acceptable agents or excipients include, without limitation, sugars, such as mannitol, dextrose, and lactose, inorganic salts, such as sodium chloride and aluminum silicates, modified starches, preservatives, antioxidants, surfactants, viscosity enhancers, coloring agents, flavoring agents, pH modifiers, sweeteners, taste-masking agents, and combinations thereof. Suitable coloring agents can include red, black and yellow iron oxides and FD & C dyes such as FD & C Blue No. 2 and FD & C Red No. 40, and combinations thereof. Suitable flavoring agents can include mint (e.g., flavour mint), raspberry, licorice, orange, lemon, grapefruit, caramel, vanilla, cherry and grape flavors and combinations of these.

In some embodiments, the pharmaceutical formulation can include about 0.001-5 wt. %, 0.01-3 wt. %, about 0.01-0.5 wt. %, about 0.02-0.4 wt. %, about 0.03-0.4 wt. %, or about 0.06 wt. % flavoring agent. In some embodiments, the pharmaceutical formulation can include at least about 0.001 wt. %, at least about 0.01 wt. %, at least about 0.02 wt. %, at least about 0.06 wt. %, or at least about 0.1 wt. % flavoring agent. In some embodiments, the pharmaceutical formulation can include at most about 5 wt. %, at most about 3 wt. %, at most about 1 wt. %, at most about 0.5 wt. %, at most about 0.1 wt. %, at most about 0.06 wt. %, at most about 0.05 wt. %, or at most about 0.01 wt. % flavoring agent.

Suitable pH modifiers can include citric acid, tartaric acid, phosphoric acid, hydrochloric acid, maleic acid, sodium hydroxide (e.g., 3 wt. % sodium hydroxide solution), and combinations thereof. In some embodiments, the pharmaceutical formulation has an amount of a pH modifier to maintain a target pH of about 2-8, about 3-7, about 3.5-6, about 3-5, or about 4.6. In some embodiments, the pH modifier is citric acid or sodium hydroxide. In some embodiments, the pharmaceutical formulation includes about 0.001-5 wt. %, about 0.01-5 wt. %, about 0.1-3 wt. %, about 2-3 wt. %, about 0.03-0.15 wt. %, or about 0.03-0.12 pH modifier. In some embodiments, the pharmaceutical formulation can include at least about 0.001 wt. %, at least about 0.002 wt. %, at least about 0.006 wt. %, or at least about 0.03 wt. % pH modifier. In some embodiments, the pharmaceutical formulation can include at most about 5 wt. %, at most about 1 wt. %, at most about 0.15 wt. %, at most about 0.12 wt. %, at most about 0.06 wt. %, or at most about 0.001 wt. % pH modifier. In some embodiments, the pH modifier can be a pH modifier solution.

In some embodiments, the pharmaceutical formulation may also include a solvent. In some embodiments, the solvent can be water (e.g., purified water). In some embodiments, the pharmaceutical formulation includes an amount of solvent such that the pharmaceutical formulation is q.s. 100%.

At step 102, the dosed pharmaceutical formulation may be frozen in the preformed molds. The dosed formulations in the preformed molds can be frozen by any means known in the art. For example, the formulations can be passed through a cryogenic chamber (e.g., liquid nitrogen tunnel). The temperature during freezing can be between about −40 to ˜120° C., about −60 to −80° C., about −65 to −75° C., or about −70° C. The freezing duration can range from about 1.5-5 minutes, about 2-4.5 minutes, about 2.5-4 minutes, about 3-4 minutes, about 3-3.5 minutes, or about 3.25 minutes. For example, the dosed formulation can be frozen at −70° C. with a residence time of 3 mins and 15 seconds. The frozen units in the preformed molds can be collected and placed in a freezer at a temperature of less than about −25° C., about −20° C., about −15° C., about −10° C., about −5° C. prior to freeze drying.

At step 103, the frozen pharmaceutical formulation may be freeze-dried to form the pharmaceutical composition. During the freeze-drying process, the water is sublimated from the frozen units. In some embodiments, the frozen units can be loaded onto the shelves of a freeze-drier. In some embodiments, the freeze-drier shelves can be precooled to below −25° C. Once the frozen units are in the freeze-drier, the freeze-drying cycle can be initiated. In some embodiments, a vacuum can be pulled and the shelf temperature raised once the freeze-drying cycle is initiated. The freeze-drier can operate at low pressure (i.e., vacuum). In some embodiments, the freeze-drier can operate at a pressure of about less than or equal to 1000 μbar, about 900 μbar, about 800 μbar, about 700 μbar, about 600 μbar, about 500 μbar, or about 400 μbar. The freeze-drying step can include holding the frozen units at the above the precooled temperatures (e.g., about −25 to 25° C., about −15 to 15° C., about −10 to 10° C., about −5 to 5° C., or about 0° C.) for about 1-24 hours, about 2-8 hours, about 4-8 hours, about 5-7 hours, or about 6 hours. In some embodiments, the units are freeze-dried at about 0° C. for about 6 hours. In some embodiments, the freeze-dried pharmaceutical compositions can be removed from the freeze-drier and inspected for any defects.

On completion of the freeze-drying cycle, the pharmaceutical compositions can be sealed (e.g., lidding foil applied to blister). The water in the freeze-dried pharmaceutical compositions can be removed via sublimation during freeze-drying. In some embodiments, the resulting pharmaceutical composition may include about 1-6 wt. %, about 1-5 wt. %, about 2-5, or about 2.5-4.75 wt. % water. In some embodiments, the water content of the pharmaceutical composition can be at most about 6 wt. %, at most about 5 wt. %, at most about 4.75 wt. %, at most about 4.5 wt. %, at most about 4 wt. %, at most about 3.5 wt. %, at most about 3 wt. %, at most about 2.5 wt. %, at most about 2 wt. %, at most about 1.5 wt. %, or at most about 1 wt. %. In some embodiments, the water content of the pharmaceutical composition can be at least about 0.01 wt. %, at least about 0.5 wt. %, at least about 1 wt. %, at least about 1.5 wt. %, at least about 2 wt. %, at least about 2.5 wt. %, at least about 3 wt. %, at least about 3.5 wt. %, or at least about 4 wt. %. In some embodiments, the water content of the pharmaceutical compositions disclosed herein are measured when the pharmaceutical compositions were made, after 3 months under storage conditions of 25° C. with 60% RH or 40° C. with 75% RH, after 6 months under storage conditions of 25° C. with 60% RH or 40° C. with 75% RH, or after 12 months under storage conditions of 25° C. with 60% RH and/or 40° C. with 75% RH.

In some embodiments, the resulting pharmaceutical composition is an ODT configured to dissolve in less than 1 minute, less than 30 seconds, or less than 10 seconds.

The pharmaceutical compositions of the present disclosure can be dissolving pharmaceutical compositions and accordingly have the distinct advantage of a faster disintegrating time.

In some embodiments, the pharmaceutical composition (e.g., post lyophilization) can include about 1-55 wt. %, about 10-50 wt. %, about 10-45 wt. %, about 10-42 wt. %, about 15-42 wt. %, about 20-42 wt. %, or about 25-35 wt. % non-gelatin matrix former. In some embodiments, the pharmaceutical composition (e.g., post lyophlization) can include about 1-10 wt. % or about 1-5 wt. % non-gelatin matrix former. In some embodiments, the pharmaceutical composition can include at least about 1 wt. %, at least about 3 wt. %, at least about 10 wt. %, at least about 15 wt. %, at least about 20 wt. %, at least about 25 wt. %, or at least about 30 wt. % non-gelatin matrix former. In some embodiments, the pharmaceutical composition can include at most about 65 wt. %, at most about 60 wt. %, at most about 55 wt. %, at most about 50 wt. %, at most about 45 wt. %, at most about 40 wt. %, at most about 35 wt. %, at most about 30 wt. %, at most about 25 wt. %, at most about 20 wt. %, at most about 15 wt. %, at most about 10 wt. %, or at most about 5 wt. % non-gelatin matrix former.

In some embodiments, the pharmaceutical composition (e.g., post lyophilization) includes about 1-50 wt. %, about 10-50 wt. %, about 10-45 wt. %, about 10-40 wt. %, about 10-35 wt. %, about 15-30 wt. %, about 20-45, about 20-43 wt. %, or about 24-42 wt. %, or about 20-35 wt. % structure former. In some embodiments, the pharmaceutical composition (e.g., post lyophilization) can include about 1-10 wt. % or about 1-5 wt. % structure former. In some embodiments, the pharmaceutical composition can include at least about 1 wt. %, at least about 3 wt. %, at least about 5 wt. %, at least about 10 wt. %, at least about 15 wt. %, at least about 20 wt. %, at least about 25 wt. %, at least about 30 wt. %, or at least about 35 wt. % structure former. In some embodiments, the pharmaceutical composition can include at most about 50 wt. %, at most about 45 wt. %, at most about 40 wt. %, at most about 35 wt. %, at most about 30 wt. %, at most about 25 wt. %, at most about 20 wt. %, at most about 15 wt. %, at most about 10 wt. %, or at most about 5 wt. % structure former.

In some embodiments, the pharmaceutical composition (e.g., post lyophilization) can include about 1-95 wt. %, about 1-75 wt. %, about 10-70 wt. %, about 15-70 wt. %, about 20-65 wt. %, about 25-65 wt. %, about 25-35 wt. % API, about 29-35 wt. %, or about 30-35 wt. % API (e.g., an antihistamine such as Loratadine). In some embodiments, the pharmaceutical composition (e.g., post lyophilization) can include about 10-90 wt. %, about 30-70 wt. %, about 40-60 wt. %, about 45-55 wt. %, or about 45-50 wt. % API (e.g., Ibuprofen Lysine). In some embodiments, the pharmaceutical composition (e.g., post lyophilization) can include about 75-99 wt. %, about 80-99 wt. %, about 85-99 wt. %, about 85-95 wt. %, or about 90-95 wt. % API (e.g., Ibuprofen). In some embodiments, the pharmaceutical composition includes at least about 1 wt. %, at least 5 wt. %, at least 10 wt. %, at least 15 wt. %, at least 20 wt. %, at least 25 wt. %, at least 30 wt. % API, at least about 35 wt. %, at least about 40 wt. %, at least about 45 wt. %, at least about 50 wt. %, at least about 75 wt. %, at least about 80 wt. %, at least about 85 wt. %, or at least about 90 wt. %. In some embodiments, the pharmaceutical composition includes at most about 95 wt. %, at most about 90 wt. %, at most about 85 wt. %, at most about 80 wt. %, at most about 75 wt. %, at most about 70 wt. %, at most about 65 wt. %, at most about 60 wt. %, at most about 55 wt. %, at most about 50 wt. % API, at most about 35 wt. % API, or at most about 30 wt. % API.

The pharmaceutical composition may also contain additional pharmaceutically acceptable agents or excipients. Such additional pharmaceutically acceptable agents or excipients include, without limitation, sugars, such as mannitol, dextrose, and lactose, inorganic salts, such as sodium chloride and aluminum silicates, modified starches, preservatives, antioxidants, surfactants, viscosity enhancers, coloring agents, flavoring agents, pH modifiers, sweeteners, taste-masking agents, and combinations thereof. Suitable coloring agents can include red, black and yellow iron oxides and FD & C dyes such as FD & C Blue No. 2 and FD & C Red No. 40, and combinations thereof. Suitable flavoring agents can include mint (e.g., flavour mint), raspberry, licorice, orange, lemon, grapefruit, caramel, vanilla, cherry and grape flavors and combinations of these. In some embodiments, the flavoring agent is flavour mint.

In some embodiments, the pharmaceutical composition (e.g., post lyophilization) includes about 0.0001-5 wt. %, about 0.0005-3 wt. %, about 0.0001-2 wt. %, about 0.001-1 wt. %, about 0.1-1 wt. %, 0.25-0.75 wt. %, or about 0.4-0.55 wt. % flavoring agent. In some embodiments, the pharmaceutical composition can include at least about 0.001 wt. %, at least about 0.01 wt. %, at least about 0.1 wt. %, at least about 0.25 wt. %, or at least about 0.4 wt. % flavoring agent. In some embodiments, the pharmaceutical composition can include at most about 5 wt. %, at most about 3 wt. %, at most about 1 wt. %, at most about 0.75 wt. %, at most about 0.55 wt. %, or at most about 0.1 wt. % flavoring agent.

In some embodiments, the pharmaceutical composition (e.g., post lyophilization) includes about 0.0001-5 wt. %, about 0.0005-3 wt. %, about 0.0001-2 wt. %, about 0.001-1 wt. %, about 0.1-2 wt. %, about 0.1-1 wt. %, or about 0.2-1 wt. % pH modifier. In some embodiments, the pharmaceutical composition can include about 0.03-0.1 wt. % pH modifier. In some embodiments, the pharmaceutical composition can include at least about 0.001 wt. %, at least about 0.01 wt. %, at least about 0.03 wt. %, at least about 0.06 wt. %, at least about 0.1 wt. %, at least about 0.2 wt. %, or at least about 0.5 wt. % pH modifier. In some embodiments, the pharmaceutical composition can include at most about 5 wt. %, at most about 3 wt. %, at most about 2 wt. %, at most about 1 wt. %, at most about 0.75 wt. %, at most about 0.5 wt. %, at most about 0.25 wt. % pH modifier, or at most about 0.1 wt. %. In some embodiments, the pH modifier in the pharmaceutical composition is just the pH modifier itself even if the pH modifier was in solution in the pharmaceutical formulation form. In some embodiments, the freeze-drying process can remove the water from the pH modifier solution.

The pharmaceutical composition may also include a solvent. In some embodiments, the solvent can be water (e.g., purified water). In some embodiments, the pharmaceutical composition (e.g., post lyophilization) includes about 0.0001-5 wt. %, about 0.0001-2 wt. %, about 0.0005-3 wt. %, or about 0.001-1 wt. % water. In some embodiments, the pharmaceutical composition can include at most about 5 wt. %, at most about 4.75 wt. %, at most about 4.5 wt. %, at most about 4 wt. %, at most about 3.5 wt. %, at most about 3 wt. %, at most about 2 wt. %, at most about 1 wt. %, at most about 0.1 wt. %, at most about 0.01 wt. %, or at most about 0.001 wt. % water. In some embodiments, the pharmaceutical composition has a low water content. In some embodiments, the pharmaceutical composition has about 1-5 wt. %, about 1-7 wt. %, about 2-6 wt. %, about 2.5-4.75 wt. %, or about at most 5% water. In some embodiments, the pharmaceutical composition has at most about 4.5 wt. % water. In some embodiments, the water content of the pharmaceutical compositions disclosed herein are measured when the pharmaceutical compositions were made, after 3 months under storage conditions of 25° C. with 60% RH or 40° C. with 75% RH, after 6 months under storage conditions of 25° C. with 60% RH or 40° C. with 75% RH, or after 12 months under storage conditions of 25° C. with 60% RH and/or 40° C. with 75% RH.

In some embodiments, the pharmaceutical composition is an ODT that can disintegrate quickly (e.g., less than 30 seconds, less than 10 seconds, less than 7 seconds, less than 3 seconds).

### FORMULATIONS, TEST METHODS, AND EXAMPLES

Several analytical studies were conducted on non-gelatin pharmaceutical compositions using a variety of non-gelatin matrix formers with the objective of comparing the properties of the non-gelatin compositions. Pharmaceutical compositions with gelatin were used as control samples for comparative assessment. The objective was also to study other characteristics of the non-gelatin compositions.

In order to study the characteristics of non-gelatin matrix formers, batches of pharmaceutical compositions with non-gelatin matrix formers were produced and studied, each with a different non-gelatin matrix former. Several tests were then performed on these batches with the aim of at least:
Identifying suitable non-gelatin matrix formers (e.g., HPMC, MC, PVA, and Pullulan) and creating lyophilized pharmaceutical compositions using the different matrix formers;
Assessing the appearance, composite assay, and purity of the non-gelatin pharmaceutical compositions;
Assessing the disintegration time of the non-gelatin pharmaceutical compositions and comparing the times obtained to gelatin pharmaceutical compositions (control);
Assessing the dissolution and stability of the non-gelatin pharmaceutical compositions and comparing the data obtained to gelatin pharmaceutical compositions (control); and/or
Assessing the water content of the non-gelatin pharmaceutical compositions and comparing the water content to gelatin pharmaceutical compositions (control).

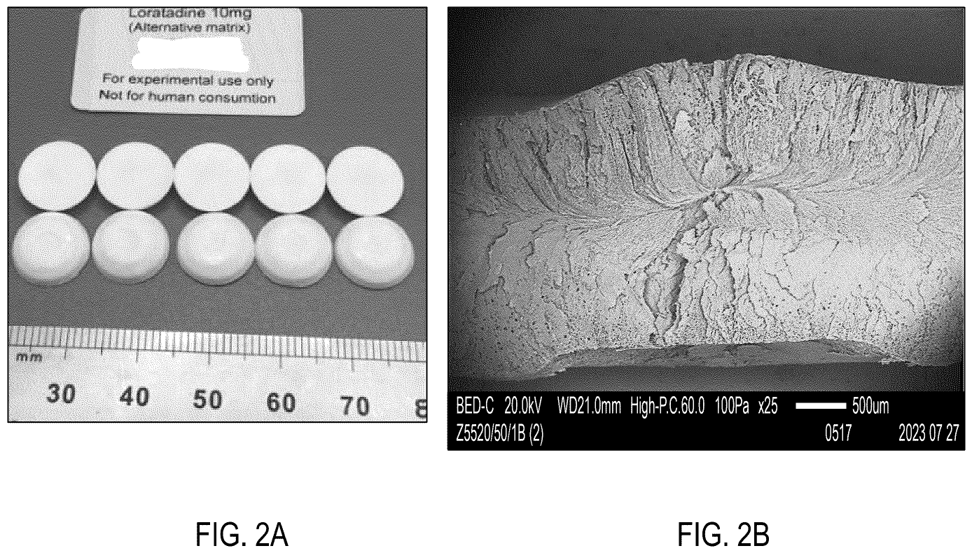

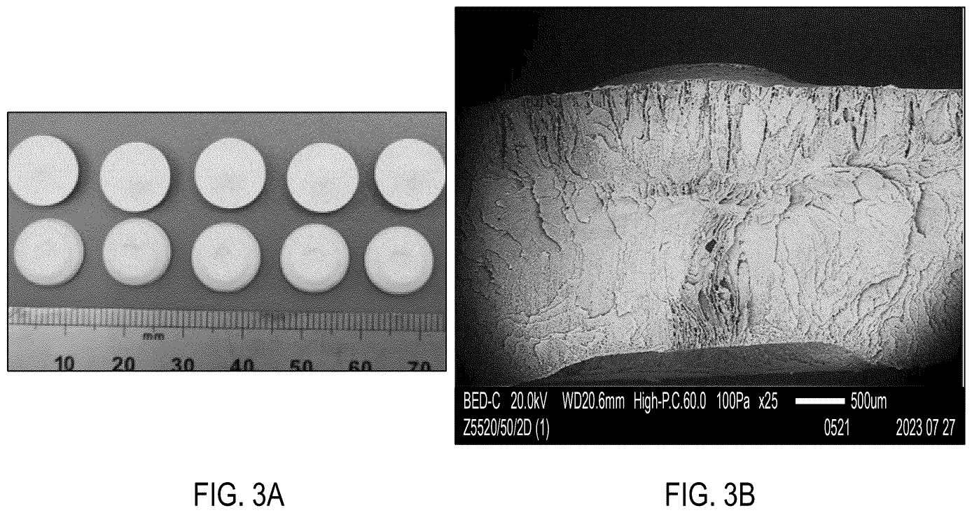

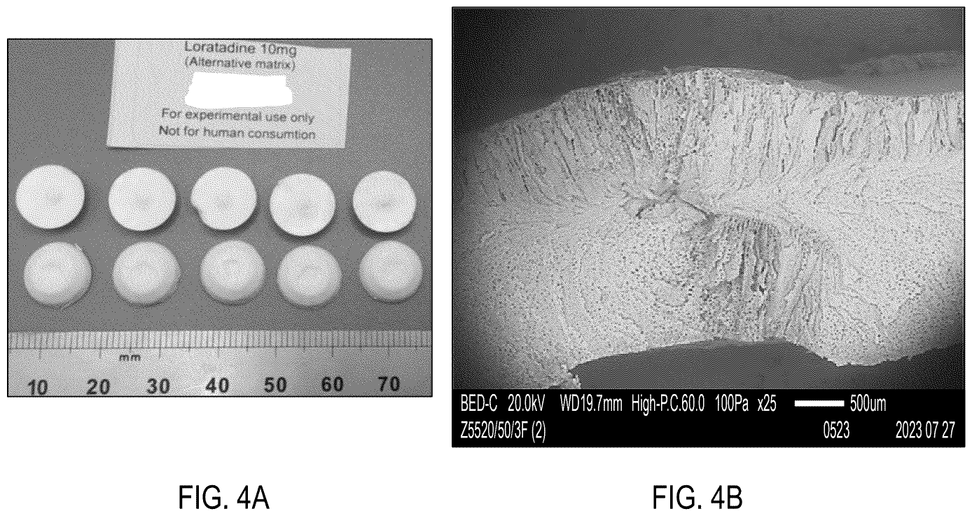

Each of the batches produced yielded a series of tablets which were studied. For example, FIG. 2A illustrates a lyophilized pharmaceutical composition using HPMC as a matrix former. FIG. 2B illustrates a cross-section of the composition using HPMC. Further, for example, FIG. 3A illustrates a lyophilized pharmaceutical composition using MC as a matrix former. FIG. 3B illustrates a cross-section of the composition using MC. Further, for example, FIG. 4A illustrates a lyophilized pharmaceutical composition using Pullulan as a matrix former. FIG. 4B illustrates a cross-section of the composition using Pullulan.

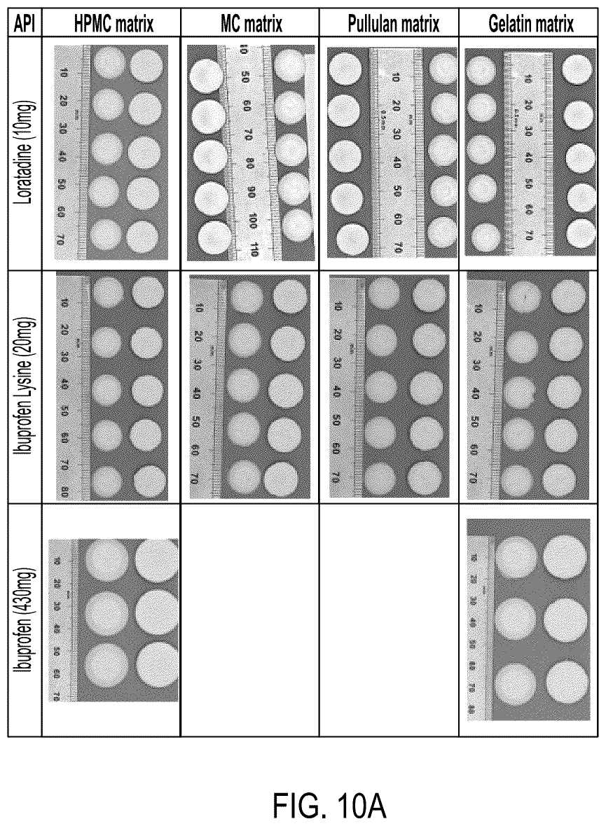

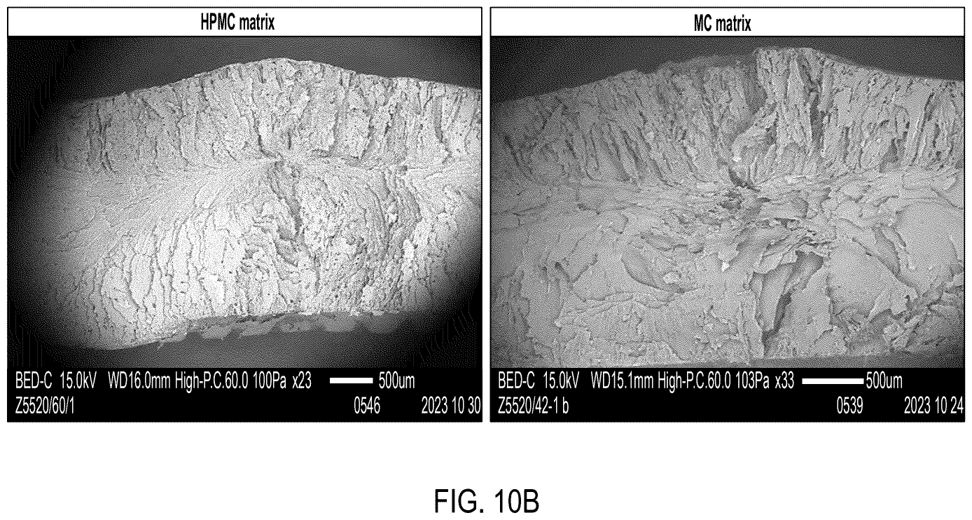

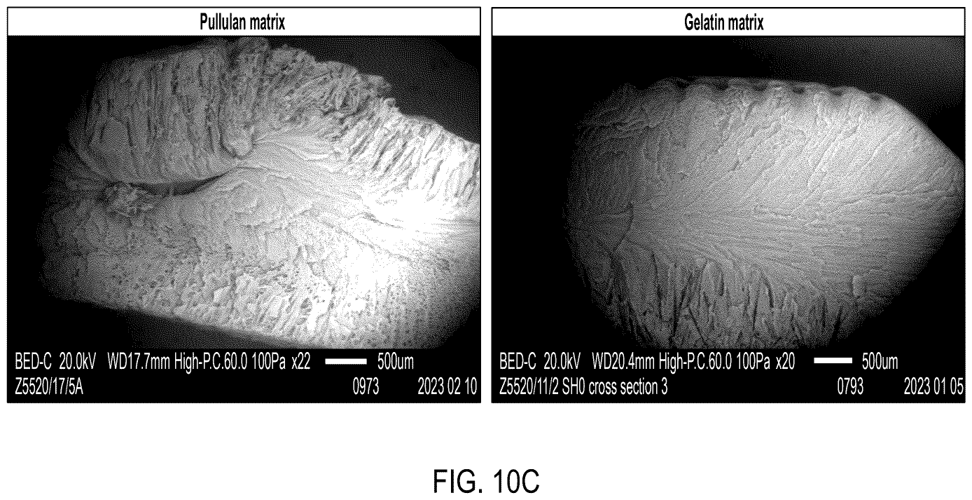

FIG. 10A illustrates the macrostructure of lyophilized pharmaceutical compositions using HPMC, MC, Pullulan, and Gelatin as the matrix former with loratadine (10 mg), Ibuprofen Lysine (20 mg), and Ibuprofen (430 mg) as the APIs. As shown in FIG. 10A, the unit appearance using Loratadine as a model drug was comparable across all matrix formers. In contrast, units containing Ibuprofen Lysine exhibited morphological defects, particularly in the Pullulan and gelatin matrix formers, which produced sunken-looking units. One gelatin-based unit even broke upon removal from the blister. These issues were not observed in units made with synthetic matrix formers such as HPMC and MC, which yielded compliant and intact units. Notably, HPMC and MC can offer superior mechanical integrity and moisture resistance, making them well-suited for formulations with higher (greater than or equal to 20 mg) and very high (˜325-430 mg) API loads and/or challenging drug-excipient interactions. The failure of the Pullulan and gelatin matrix formers may be attributed to poor compatibility between the model drug and the selected matrix formers, or to excessive API loading, which likely led to structural defects. FIGS. 10B-10C illustrate a cross-section of a lyophilized pharmaceutical composition (i.e., the microstructure) using HPMC, MC, Pullulan, and Gelatin as a matrix former without an API (i.e., a placebo).

The following Tables 1A, 1B, and 1C include example pharmaceutical formulations (before freeze drying) used in the studies described herein and Table 1D includes the control gelatin-based pharmaceutical formulations (before freeze drying) described herein.

TABLE 1A

| | 1 | | 2 | | 3 | |
|---|---|---|---|---|---|---|
| | % w/w | [mg] | % w/w | [mg] | % w/w | [mg] |
| Loratadine | 4.00 | 10.0 | 4.00 | 10.0 | 4.00 | 10.0 |
| HPMC | 4.00 | 10.0 | — | — | — | — |
| (Hypromellose) | | | | | | |
| MC (Metolose) | — | — | 2.50 | 6.25 | — | — |
| Pullulan | — | — | — | — | 5.00 | 12.5 |
| GalenIQ ™ | 5.00 | 12.5 | 5.00 | 12.5 | 3.00 | 7.5 |
| Flavour mint | 0.06 | 0.15 | 0.06 | 0.15 | 0.06 | 0.15 |
| Citric acid | 0.084 | 0.21 | 0.117 | 0.29 | 0.031 | 0.08 |
| Purified water | qs. 100 | qs. 250 | qs. 100 | qs. 250 | qs. 100 | qs. 250 |

TABLE 1B

| | 1 | | 2 | | 3 | |
|---|---|---|---|---|---|---|
| | % w/w | [mg] | % w/w | [mg] | % w/w | [mg] |
| Ibuprofen | 8.00 | 20.00 | 8.00 | 20.00 | 8.00 | 20.00 |
| Lysine | | | | | | |
| HPMC | 4.00 | 10.00 | — | — | — | — |
| (Hypromellose) | | | | | | |
| MC (Metolose) | — | — | 2.50 | 6.30 | — | — |
| Pullulan | — | — | — | — | 5.00 | 12.50 |
| Mannitol | 5.00 | 12.50 | 5.00 | 12.50 | 3.00 | 7.50 |
| Purified water | qs. 100 | qs. 250 | qs. 100 | qs. 250 | qs. 100 | qs. 250 |

TABLE 1C

1

| | % w/w | [mg] |
|---|---|---|
| Ibuprofen | 43.04 | 430.40 |
| HPMC (Hypromellose) | 1.71 | 17.10 |
| Mannitol | 1.71 | 17.10 |
| Purified water | qs. 100 | qs. 1000 |

TABLE 1D

| | 1 | | 2 | | 3 | | 4 | |
|---|---|---|---|---|---|---|---|---|
| Control | % w/w | [mg] | % w/w | [mg] | % w/w | [mg] | % w/w | [mg] |
| Gelatin | 3.59 | 8.98 | 3.59 | 8.98 | 3.59 | 8.98 | 1.40 | 14.00 |
| Mannitol | 2.88 | 7.20 | 2.88 | 7.20 | 2.88 | 7.20 | 1.70 | 17.00 |
| Citric acid | 0.10 | 0.25 | 0.10 | 0.25 | N/A | N/A | N/A | N/A |
| Mint (1 & 2) | 0.06 | 0.15 | 0.06 | 0.15 | N/A | N/A | 0.02 | 0.20 |
| Xanthan Gum (4) | | | | | | | | |
| Loratadine (1 & 2) | 4.00 | 10.00 | 4.00 | 10.00 | 8.00 | 20.00 | 43.04 | 430.40 |
| Ibuprofen Lysine (3) | | | | | | | | |
| Ibuprofen (4) | | | | | | | | |
| Purified water | qs. 100 | qs. 250 | qs. 100 | qs. 250 | qs. 100 | qs. 250 | qs. 100 | qs. 1000 |

The following Tables 2A, 2B, 2C, and 2D include example pharmaceutical compositions (e.g., post-lyophilization) used in the studies described herein made from the pharmaceutical formulations described above in Tables 1A, 1B, 1C, and 1D such that the pharmaceutical compositions of Table 2A correspond with the pharmaceutical formulations of Table 1A, the pharmaceutical compositions of Table 2B correspond with the pharmaceutical formulations of Table 1B, the pharmaceutical composition of Table 2C corresponds with the pharmaceutical formulation of Table 1C, and the control pharmaceutical compositions of Table 2D correspond with the pharmaceutical formulations of Table 1D. The theoretical weight of a unit is calculated by adding the weight of the dry components excluding water as it is sublimed in the lyophilization (e.g., freeze-drying) process. The actual unit weight is higher due to bound water in the finished product, whereas the theoretical unit weight is calculated based on solid content prior to the freeze-drying process.

TABLE 2A

| | 1 | 2 | 3 |
|---|---|---|---|
| | % w/w | % w/w | % w/w |
| Loratadine | 29.18 | 34.57 | 33.16 |
| HPMC | 29.18 | — | — |
| (Hypromellose) | | | |
| MC (Metolose) | — | 21.60 | — |
| Pullulan | — | — | 41.45 |
| GalenIQ ™ | 36.48 | 43.21 | 24.87 |
| Flavour mint | 0.44 | 0.52 | 0.50 |
| Citric acid | 0.06 | 0.10 | 0.03 |
| Finished product | 32.67 | 28.93 | 33.16 |
| weight [mg] - | | | |
| Theoretical | | | |
| Finished product | 34.27 (average | 30.65 (average | 31.12 (average |
| weight [mg] - actual | of 24 units) | of 18 units) | of 24 units) |

TABLE 2B

| | 1 | 2 | 3 |
|---|---|---|---|
| | % w/w | % w/w | % w/w |
| Ibuprofen Lysine | 47.06 | 47.06 | 50.00 |
| HPMC | 29.41 | — | — |
| (Hypromellose) | | | |
| MC (Metolose) | — | 23.53 | — |
| Pullulan | — | — | 31.25 |
| Mannitol | 29.41 | 29.41 | 18.75 |
| Finished product | 42.50 | 42.50 | 40.00 |
| weight [mg] - | | | |
| Theoretical (close | | | |
| enough to actual) | | | |

TABLE 2C

| | 1 |
|---|---|
| | % w/w |
| Ibuprofen | 92.64 |
| HPMC (Hypromellose) | 3.68 |
| Mannitol | 3.68 |
| Finished product weight | 464.6 |
| [mg] - Theoretical (close | |
| enough to actual) | |

TABLE 2D

| | 1 | 2 | 3 | 4 |
|---|---|---|---|---|
| Control | % w/w | % w/w | % w/w | % w/w |
| Gelatin | 33.77 | 33.77 | 24.81 | 3.03 |
| Mannitol | 27.09 | 27.09 | 19.90 | 3.68 |
| Citric acid | 0.94 | 0.94 | N/A | N/A |
| Mint (1 & 2) | 0.56 | 0.56 | N/A | 0.04 |
| Xanthan Gum (4) | | | | |
| Loratadine (1 & 2) | 37.62 | 37.62 | 55.29 | 93.24 |
| Ibuprofen Lysine (3) | | | | |
| Ibuprofen (4) | | | | |
| Finished product | 26.58 | 26.58 | 43.41 | 461.60 |
| weight [mg] - | | | | |
| Theoretical (close | | | | |
| enough to actual) | | | | |

Tables 1A, 1B, 1C, and 1D list exemplary pharmaceutical formulations in wt. % used in the analytical studies and weights in mg of the various components in the pharmaceutical formulations. Tables 2A, 2B, 2C, and 2D list exemplary pharmaceutical compositions in wt. % used in the analytical studies. In Table 1A, Batch 1 included HPMC as a matrix former with isomalt (e.g., GalenIQ™) as the structure former, Batch 2 included MC as a matrix former with isomalt (e.g., GalenIQ™) as the structure former, and Batch 3 included Pullulan as a matrix former with isomalt (e.g., GalenIQ™) as a structure former. All three batches of Table 1A included an antihistamine (here, Loratadine) as the API and all three batches also included flavour mint as a taste enhancer, and citric acid to adjust the pH to 4.6.

Each of the pharmaceutical compositions of Tables 2A, 2B, 2C, and 2D was created by dosing a pharmaceutical formulation into a preformed mold, freezing the dosed pharmaceutical formulation, and freeze-drying the frozen pharmaceutical formulation to form the pharmaceutical composition. The units were frozen in temperature range −60° C. to −90° C. However, for lower or higher fill volumes, this temperature range could be −40° C. to −110° C. The freeze-drying could take any between 3 hours and 24 hours. The tablet wet fill volume of the pharmaceutical formulations of Tables 1A and 1B was 250 mg and for 1C was 1000 mg.

Several tablets of the pharmaceutical composition were prepared for each batch for the analytical studies, as noted in Tables 2A, 2B, and 2C. Tables 2A, 2B, and 2C also lists the average theoretical mass of the tablets after freeze-drying for each of the batches using the formulations listed in Tables 1A, 1B, and 1C, respectively. Several tablets using gelatin matrix formers were also prepared as controls, and these pharmaceutical compositions are listed in Table 2D after freeze-drying the formulations listed in Table 1D.

Several tests were conducted, each of which are disclosed herein, measuring such characteristics, for example, as: appearance, composite assay, water content, disintegration times, related compounds, and water activity.

### Appearance, Composite Assay, and Related Substances

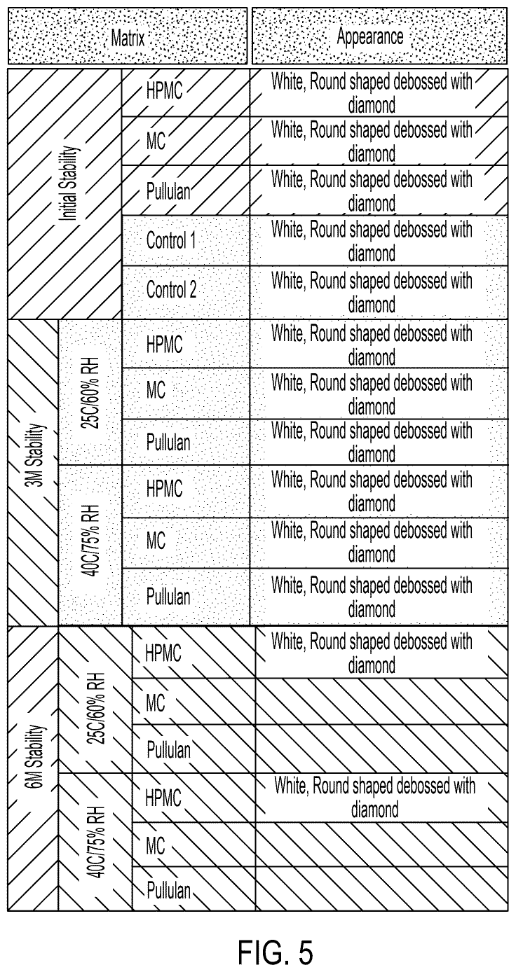

One objective of the studies was to study at least the appearance, composite assay, and related substances (impurities) for the batches of tablets being stored under stability conditions at 25° C. with 60% relative humidity (“RH”) and at 40° C. with 75% RH tested at specified time points as seen in FIG. 5 using Loratadine as a model drug. At least three notes on appearance were taken at different stability timepoints—for example, one initial timepoint (when the tablets were made), one at 3 months, and one after storage for 6 months and 12 months. For all stability conditions for each of the batches (and controls), the appearance of the tablet was white, round shaped debossed with diamond, demonstrating that the matrix former discovered herein produced acceptable product with respect to these characteristics.

The following Table 3 is the composite assay data for the pharmaceutical compositions used in the studies described herein.

TABLE 3

| | Composite Assay (n = 4), % label claim | | | | | |
|---|---|---|---|---|---|---|
| | Stability timepoints | | | | | |
| | | 3 months | | 6 months | | 12 months |
| Matrix in | | 25° C./ | 40° C./ | 25° C./ | 40° C./ | 25° C./ |
| Formulation | Initial | 60% RH | 75% RH | 60% RH | 75% RH | 60% RH |
| HPMC matrix | 105.3% | 106.1% | 105.9% | 105.4% | 106.1% | 105.5% |
| MC matrix | 106.5% | 106.5% | 106.6% | 106.6% | 106.4% | 106.7 |
| Pullulan matrix | 104.1% | 103.9% | 104.7% | 103.0% | 104.4% | 104.6 |
| Control 1 | 96.0% | — | — | — | — | 95.9% |
| Control 2 | 100.2% | — | — | — | — | — |

Model drug: Loratadine with label claim of 10 mg per tablet

As seen in Table 3, a composite assay test was performed for at least four stability timepoints—for example, one initially (when the tablets were made), one at 3 months, one at 6 months, and one at 12 months for stability conditions at 25° C. with 60% RH and at 40° C. with 75% RH using Loratadine (10 mg) as a model drug. The test was measured for two composite samples of 10 tablets, each employing HPLC analysis in duplicates. The composite sample is the average of the 4 replicates (n=4) as Composite Assay testing requires 2 samples and two HPLC injections of each. The label claim indicates the quantity of the active drug component alleged to be in each tablet. This test ensures that the quality of drug product by ensuring that the correct amount of API is added to units. For all stability conditions for each of the batches (and controls), the assay results were comparable over time and the results were within UPS Monograph specification of 90-110% (or 95-105%) of label claim (for Loratadine Tablet) demonstrating that the matrix former discovered herein produced acceptable product with respect to this characteristic.

In some embodiments, the composite assay of the pharmaceutical compositions and their corresponding APIs disclosed herein are the percent of label claim as defined in the pharmacopeia monographs per the individual active pharmaceutical ingredients as defined by USP and/or Ph. Eur. In some embodiments, the composite assay can be 90-110% or 95-105% of label claim. In some embodiments, the composite assay of the pharmaceutical compositions and their corresponding APIs disclosed herein are measured when the pharmaceutical compositions were made, after 3 months under storage conditions of 25° C. with 60% RH or 40° C. with 75% RH, after 6 months under storage conditions of 25° C. with 60% RH or 40° C. with 75% RH, and/or after 12 months under storage conditions of 25° C. with 60% RH or 40° C. with 75% RH.

The following Table 4 is the related substances results for the pharmaceutical compositions used in the studies described herein.

TABLE 4

| | Related Substances | | | | | |
|---|---|---|---|---|---|---|
| | Stability timepoints | | | | | |
| | | 3 months | | 6 months | | 12 months |
| | | 25° C./ | 40° C./ | 25° C./ | 40° C./ | 25° C./ |
| Formulation | Initial | 60% RH | 75% RH | 60% RH | 75% RH | 60% RH |
| HPMC matrix | ND | ND | ND | ND | ND | ND |
| MC matrix | ND | ND | ND | ND | ND | ND |
| Pullulan matrix | ND | ND | ND | ND | ND | ND |
| Control 1 | ND | | | | | ND |
| Control 2 | ND | | | | | |

ND = Not Detected

As seen in Table 4, a related substances test was performed for at least four stability timepoints—for example, one initial timepoint (when the tablets were made), one at 3 months, one at 6 months, and one at 12 months at 25° C. with 60% RH and at 40° C. with 75% RH using Loratadine as a model drug. The tests looked for at least specified degradation products, synthesis impurities, and unspecified impurities to ensure they do not exceed maximum specified amounts (e.g., maximum of 0.05% of degradation product, maximum of 0.2% of synthesis impurity, maximum of 0.1% of unspecified impurities). Degradation products were measured using high-performance liquid chromatography (“HPLC”) analysis. At all the observed stability timepoints for each of the batches (and controls), the tablets had no impurities, indicating relatively pure pharmaceutical compositions and demonstrating that the matrix former discovered herein produced an acceptable product with respect to this characteristic. The new matrix former materials did not contribute to formation of specified degradation products

### Disintegration Studies

The following Table 5 is the disintegration results for the pharmaceutical compositions used in the studies described herein.

TABLE 5

| | | Disintegration time, seconds | | | | | | | | | | | |
|---|---|---|---|---|---|---|---|---|---|---|---|---|---|
| | | Stability timepoints | | | | | | | | | | | |
| | | | | 3 months | | | | 6 months | | | | 12 months | |
| | Matrix in | | | 25° C./ | | 40° C./ | | 25° C./ | | 40° C./ | | 25° C./ | |
| API | Formulation | Initial | | 60% RH | | 75% RH | | 60% RH | | 75% RH | | 60% RH | |
| Loratadine | HPMC matrix | <6 | s | <4 | s | <4 | s | <4 | s | <5 | s | <4 | s |
| | MC matrix | <2 | s | <5 | s | <2 | s | <4 | s | <3 | s | <2 | s |
| | Pullulan matrix | <2 | s | <3 | s | <3 | s | <3 | s | <3 | s | <2 | s |

| | | | | | | | | |
|---|---|---|---|---|---|---|---|---|
| Control 1 | <1 | s | — | — | — | — | <2 | s |

| | | | | | | | | |
|---|---|---|---|---|---|---|---|---|
| | Control 2 | <1 | s | — | — | — | — | — |
| Ibuprofen lysine | HPMC matrix | <1 | s | — | — | — | — | — |
| | MC matrix | <1 | s | — | — | — | — | — |
| | Pullulan matrix | <1 | s | — | — | — | — | — |
| | Control 3 | <1 | s | — | — | — | — | — |
| Ibuprofen | HPMC matrix | <7 | s | — | — | — | — | — |
| | Control 4 | <6 | s | — | — | — | — | — |

Control = Gelatin matrix

One objective of the studies was to study at least the disintegration characteristics of the pharmaceutical compositions. This test may determine how quickly the pharmaceutical composition disperses in water and may estimate behavior in the buccal cavity. In some embodiments, it is desirable to create pharmaceutical compositions capable of immediate and/or prolonged release without affecting the disintegration time. As seen in Table 5, disintegration tests were performed at different periods of time—for example, one initially (when the tablets were made), one at 3 months, one at 6 months, and one at 12 months (for certain compositions). The disintegration testing was also performed at different humidities and temperatures—for example, at 25° C. with 60% RH and at 40° C. with 75% RH. In the disintegration testing, the disintegration water bath was filled with water heated to 37° C. (±2° C.). Then purified water was added to six beakers in placed in the bath until they reached 37° C. The volume of water in the beaker is such that, when the backet-rack assembly is in the highest position, the wire mesh is at least 15 mm below the surface of the water in the disintegration bath. The basket-rack assembly comprises six tube-like chambers where individual units are placed with a stainless clip attached to them to ensure the units are immersed in water. In all trials, disintegration of the pharmaceutical composition did not take longer than 30 seconds. More specifically, the mean disintegration times were all less than 10 seconds (and most less than 5 seconds). Further, the disintegration time of the non-gelatin pharmaceutical compositions compared favorably with the control (gelatin) pharmaceutical compositions. Specifically, the non-gelatin pharmaceutical compositions had disintegration times that averaged not more than a few seconds more than the control pharmaceutical compositions.

As previously mentioned, disintegration studies were performed at multiple stability timepoints. For example, initial disintegration studies of the pharmaceutical compositions were performed using HPMC, MC, Pullulan, and gelatin as matrix formers, respectively. Each was placed in a disintegration tester to measure the amount of time it took for the pharmaceutical composition to disintegrate. Then, for example, the pharmaceutical compositions were tested after being stored at stability conditions for 3 and 6 months at 25° C./60% RH and 40° C./75% RH which may be indicative of pharmaceutical composition stability over time in varying conditions. The 3-month disintegration studies of the pharmaceutical compositions were performed using MC as matrix former at two stability conditions of 25° C. at 60% RH and 40° C. at 75% RH, yielding an average disintegration time of 5 s and 2 s, respectively. The 3-month disintegration studies of the pharmaceutical compositions were also performed using Pullulan as matrix former at two stability conditions of 25° C. at 60% RH and 40° C. at 75% RH, yielding an average disintegration time of 3 s and 3 s, respectively. Then, for example, the 6-month disintegration studies of the pharmaceutical compositions were performed using HPMC as matrix former at two stability conditions of 25° C. at 60% RH and 40° C. at 75% RH, yielding an average disintegration time of 4 s and 5 s, respectively.

These studies may indicate that using these alternative, non-animal origin (non-gelatin) matrix formers either alone or in combination, it is possible to produce ODTs that do not affect the disintegration characteristics requirement of the ODTs (e.g., disintegration times less than 30 s).

In some embodiments, the disintegration times of the pharmaceutical compositions disclosed herein are less than or equal to about 30 seconds, less than or equal to 25 seconds, less than or equal to 20 seconds, less than or equal to 15 seconds, less than or equal to 10 seconds, less than or equal to 7 seconds, less than or equal to 6 seconds, less than or equal to 5 seconds, less than or equal to 4 seconds, less than or equal to 3 seconds, less than or equal to 2 seconds, or less than or equal to 1 second. In some embodiments, the disintegration times of the pharmaceutical compositions disclosed herein are greater than or equal to 0.5 seconds, greater than or equal to 1 second, greater than or equal to 1.5 seconds, greater than or equal to 2 seconds, greater than or equal to 2.5 seconds, greater than or equal to 3 seconds, greater than or equal to 3.5 seconds, greater than or equal to 4 seconds, greater than or equal to 4.5 seconds, or greater than or equal to 5 seconds. In some embodiments, the disintegration times of the pharmaceutical compositions disclosed herein can be about 0.5-7 seconds, about 1-7 seconds, about 1-6 second, or about 1-5 seconds. In some embodiments, the disintegration times of the pharmaceutical compositions disclosed herein are measured when the pharmaceutical compositions were made, after 3 months under storage conditions of 25° C. with 60% RH or 40° C. with 75% RH, after 6 months under storage conditions of 25° C. with 60% RH or 40° C. with 75% RH, and/or after 12 months under storage conditions of 25° C. with 60% RH or 40° C. with 75% RH.

### Dissolution Studies

One objective of the studies was to study at least the dissolution characteristics of the pharmaceutical compositions. In some embodiments, it is desirable to create non-animal origin pharmaceutical compositions with at least comparable drug dissolution profiles compared to gelatin pharmaceutical compositions. The following Dissolution Table contains dissolution test results for the pharmaceutical compositions used in the studies described herein.

| | Dissolution (at 6 min), % release of Label Claim | | | | | |
|---|---|---|---|---|---|---|
| | Stability timepoints | | | | | |
| | | 3 months | | 6 months | | 12 months |
| Matrix in | | 25° C./ | 40° C./ | 25° C./ | 40° C./ | 25° C./ |
| Formulation | Initial | 60% RH | 75% RH | 60% RH | 75% RH | 60% RH |
| HPMC matrix | 102.7% | 104.5% | 104.9% | 104.7% | 101.3% | 91.9% |
| MC matrix | N/A | N/A | N/A | N/A | N/A | 102.4% |
| Pullulan matrix | 92.8% | 94.4% | 96.6% | 101.8% | 97.1% | 91.4% |
| Control 1 | 94.4% | — | — | — | — | 91.4 |
| Control 2 | 99.1% | — | — | — | — | — |

Model drug: Loratadine

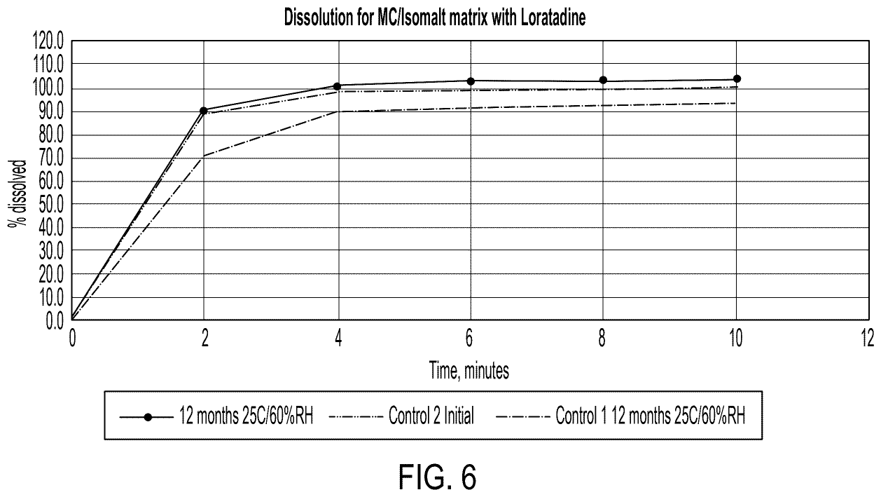

As seen in FIGS. 6-8 dissolution tests were performed at different stability timepoints—for example, one initial timepoint (when the tablets were made), one at 3 months, one at 6 months, and one at 12 months at two stability conditions of 25° C. at 60% RH and 40° C. at 75% RH. Each test tested, for example, the percent dissolution of the pharmaceutical composition over time, measuring the drug release over time where the model drug was Loratadine. The dissolution tests were performed by an automated dissolution system with ultraviolet absorbance detection employing basket apparatus as per USP <711>. The specification for Loratadine is 80% release at 6 minutes as per USP for ODT.

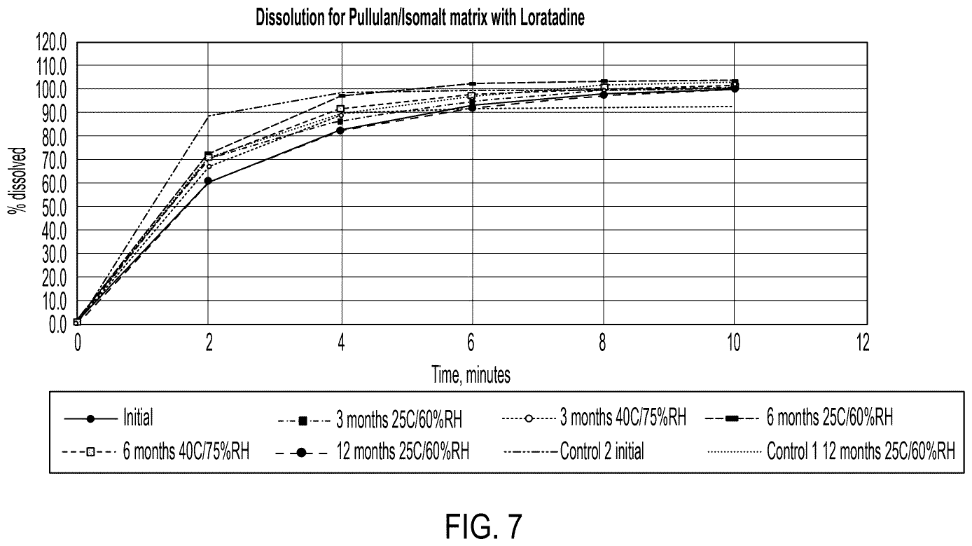

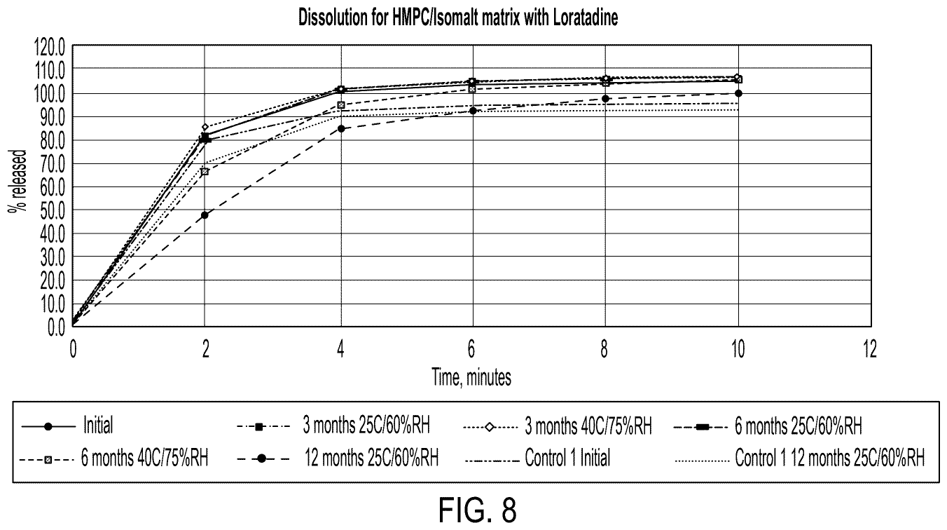

FIG. 6 shows the dissolution study of the pharmaceutical compositions using MC as matrix former at the 12 month timepoint. FIG. 7 shows the dissolution study of the pharmaceutical compositions using Pullulan as matrix former with the 3, 6, and 12 month timepoint. FIG. 8 shows the dissolution study of the pharmaceutical compositions using HPMC as matrix former with the 3, 6, and 12 month timepoint.

These studies may indicate that ODTs with HPMC and Pullulan gave drug dissolution profiles comparable with the control (gelatin matrix former). This work may indicate that using these alternative, non-animal origin matrix formers either alone or in combination, make it possible to produce ODTs with drug dissolution profiles equivalent to those with gelatin.

In some embodiments, the percent dissolution of the pharmaceutical compositions and their corresponding API disclosed herein after 6 minutes is at least about 75%, at least about 80%, at least about 85%, at least about 90%, at least about 95%, or at least about 100% of label claim. In some embodiments, the percent dissolution of the pharmaceutical compositions and their corresponding API disclosed herein after 6 minutes is at most about 110%, at most about 105%, at most about 100%, at most about 95%, or at most about 90% of label claim. In some embodiments, the percent dissolution of the pharmaceutical compositions and their corresponding API disclosed herein after 6 minutes is about 90-110% of label claim. In some embodiments, the percent dissolution of the pharmaceutical compositions and their corresponding API disclosed herein are measured when the pharmaceutical compositions were made, after 3 months under storage conditions of 25° C. with 60% RH or 40° C. with 75% RH, after 6 months under storage conditions of 25° C. with 60% RH or 40° C. with 75% RH, and/or after 12 months under storage conditions of 25° C. with 60% RH or 40° C. with 75% RH.

### Water Content Studies

The following Table 6 is the water activity results for the pharmaceutical compositions used in the studies described herein.

TABLE 6

| | Water Activity, ERH | | | | | |
|---|---|---|---|---|---|---|
| | Stability timepoints | | | | | |
| | | 3 months | | 6 months | | 12 months |
| | | 25° C./ | 40° C./ | 25° C./ | 40° C./ | 25° C./ |
| Matrix in Formulation | Initial | 60% RH | 75% RH | 60% RH | 75% RH | 60% RH |
| HPMC matrix former | 0.40 | 0.23 | 0.23 | 0.26 | 0.28 | 0.35 |
| MC matrix former | 0.43 | 0.35 | 0.34 | 0.38 | 0.38 | 0.23 |
| Pullulan matrix former | 0.43 | 0.39 | 0.36 | 0.43 | 0.42 | 0.25 |
| Control 1 | 0.43 | — | — | — | — | 0.47 |
| Control 2 | 0.41 | — | — | — | — | — |

Model drug: Loratadine

One objective of the studies was to study at least the water content characteristics of the pharmaceutical compositions. In some embodiments, it is desirable to have pharmaceutical compositions with low water content for improved ODT product stability during storage, less stringent requirements for moisture protective packaging, and/or greater stability for moisture sensitive APIs. As seen in Table 6, at least four water activity tests were performed at different stability timepoints—for example, one initial timepoint (when the tablets were made), one at 3 months, one at 6 months, and one at 12 months for two stability conditions 25° C. with 60% RH and at 40° C. with 75% RH. The water activities were measured as the equilibrium relative humidity (“ERH”). In all of the studies, the water activity was not more than 0.6. This may indicate that the pharmaceutical compositions have low water activity and thus low water content, a desirable characteristic for ODTs. Water activity is a pharmacopeial test with a reference of USP <922> and the typical specification is less than 0.6 which indicates that microbial growth is unlikely due to low water activity.

In some embodiments, the water activity of the pharmaceutical compositions disclosed herein is about 0.1-0.5 or about 0.2-0.45. In some embodiments, the water activity of the pharmaceutical compositions disclosed herein is at least about 0.05, at least about 0.1, at least about 0.15, at least about 0.2, at least about 0.25, at least about 0.3, at least about 0.35, or at least about 0.4. In some embodiments, the water activity of the pharmaceutical compositions disclosed herein is at most about 0.5, at most about 0.45, at most about 0.4, at most about 0.35, at most about 0.3, or at most about 0.25. In some embodiments, the water activity of the pharmaceutical compositions disclosed herein are measured when the pharmaceutical compositions were made, after 3 months under storage conditions of 25° C. with 60% RH or 40° C. with 75% RH, after 6 months under storage conditions of 25° C. with 60% RH or 40° C. with 75% RH, or after 12 months under storage conditions of 25° C. with 60% RH and/or 40° C. with 75% RH.

The following Table 7 is the water content results for the pharmaceutical compositions used in the studies described herein.

TABLE 7

| | Water content (n = 10), % w/w | | | | | |
|---|---|---|---|---|---|---|
| | Stability timepoints | | | | | |
| | | 3 months | | 6 months | | 12 months |
| Matrix in | | 25° C./ | 40° C./ | 25° C./ | 40° C./ | 25° C./ |
| Formulation | Initial | 60% RH | 75% RH | 60% RH | 75% RH | 60% RH |
| HPMC matrix | 3.18% | 3.71% | 3.24% | 3.74% | 3.51% | 3.16% |
| MC matrix | 3.17% | 3.97% | 4.34% | 3.02% | 3.73% | 2.69% |
| Pullulan matrix | 3.90% | 4.37% | 4.64% | 3.86% | 4.56% | 3.56% |
| Control 1 | 4.19% | — | — | — | — | 4.06% |
| Control 2 | 3.74% | — | — | — | — | — |

Model drug: Loratadine
10 tablets are tested for water content and data in the table is for the mean of 10.

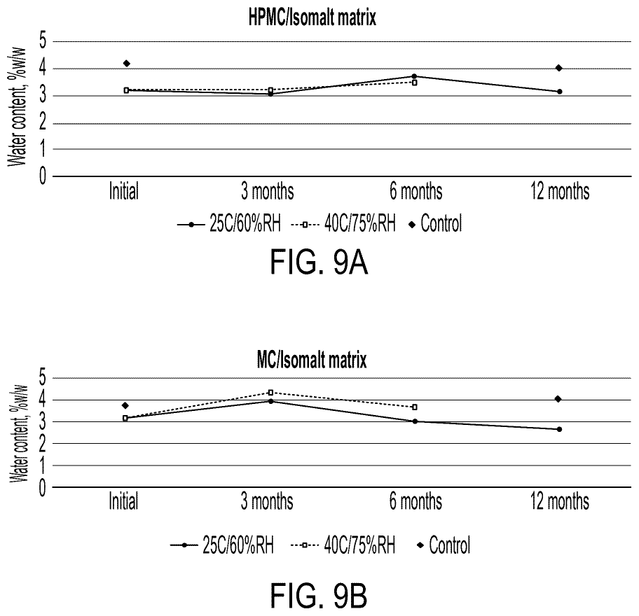

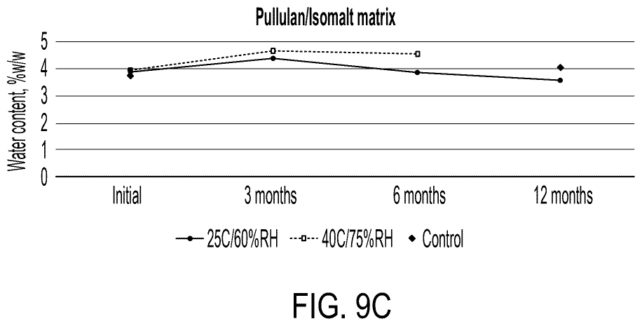

Further water content studies were performed, for example, by using a volumetric Karl Fisher titrator. As seen in Table 7 and FIGS. 9A-C, at least four water content tests (in wt. %) were performed at different timepoints—for example, one initial timepoint (when the tablets were made), one at 3 months, one at 6 months, and one at 12 months for two stability conditions 25° C. with 60% RH and at 40° C. with 75% RH. It was observed that the mean water content for most pharmaceutical compositions studied was not more than 5 wt. %. Water content was tested per USP <921>.

The initial water content for all tested matrix formers, including hydroxypropyl methylcellulose (HPMC), methylcellulose (MC), and Pullulan, was 3.2%, 3.2% and 3.9% respectively, whereas the average of two gelatin matrix former units was 4.0%.

After 12 months, the HPMC and MC matrix former units demonstrated superior moisture stability, maintaining water content of ˜3% w/w. Pullulan matrix former units exhibited slightly higher water retention, with water content values approaching 3.6% w/w. In contrast, the gelatin matrix former unit water content exceeded 4% w/w.

These results indicate that the HPMC and MC matrix formers can provide enhanced resistance to moisture uptake over time, contributing to improved physical and chemical stability of the composition. The reduced water retention observed in HPMC formulations is consistent with its reversible moisture sorption behavior and moderate porosity, as confirmed by Dynamic Vapor Sorption (DVS) analysis.

These studies may indicate low water content which can lead to better stability in humid environments, lower likelihood of microbial growth, better resistance to polymorphic/pseudo-polymorphic changes in water-sensitive APIs, and/or less stringent need for hermetic packaging.

### Dynamic Vapour Sorption Studies

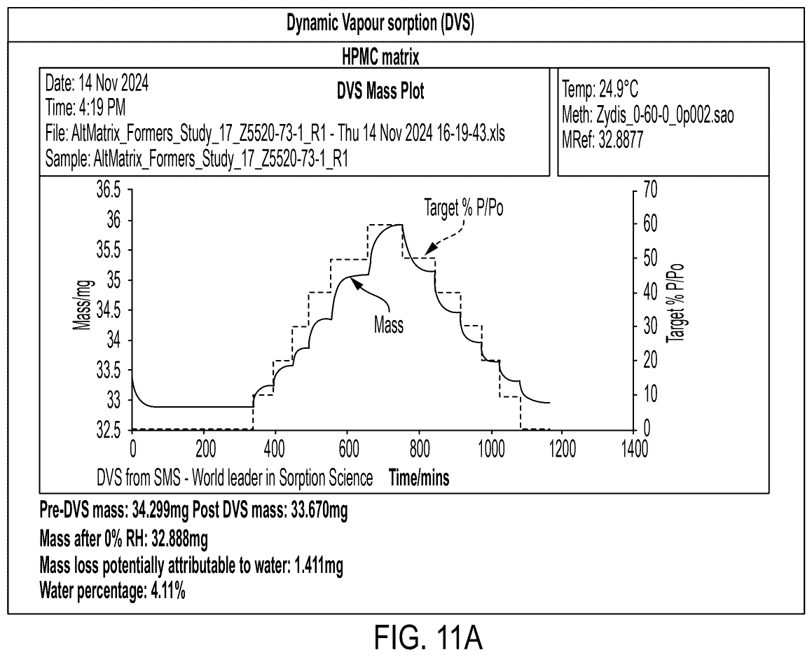

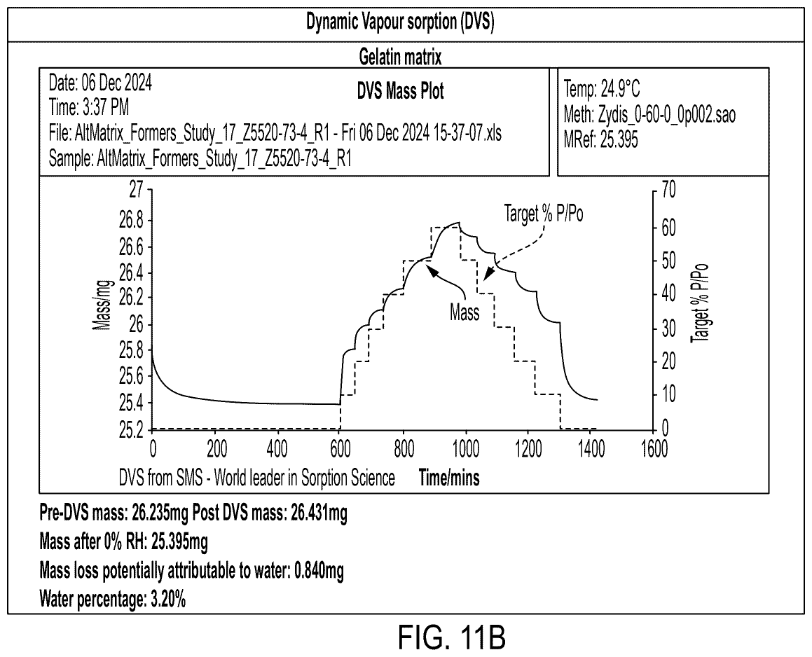

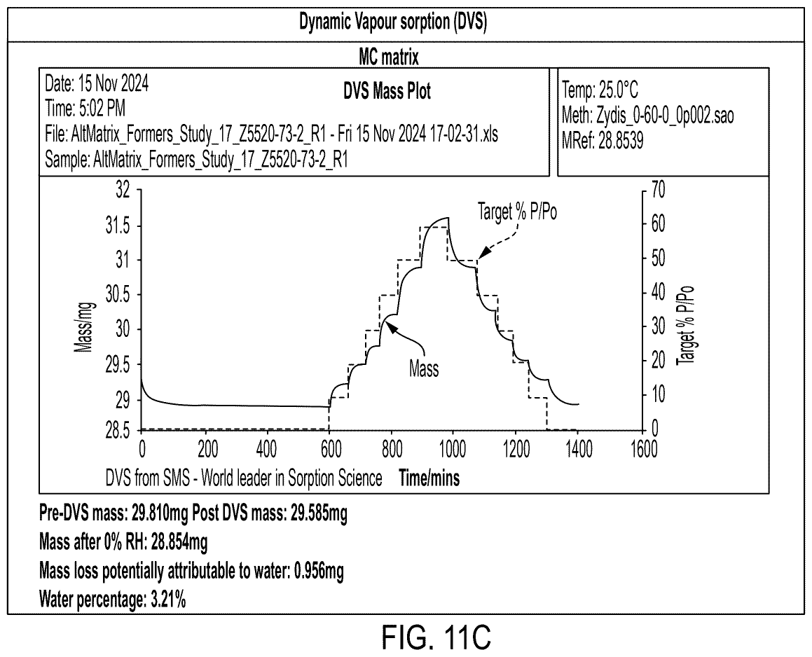

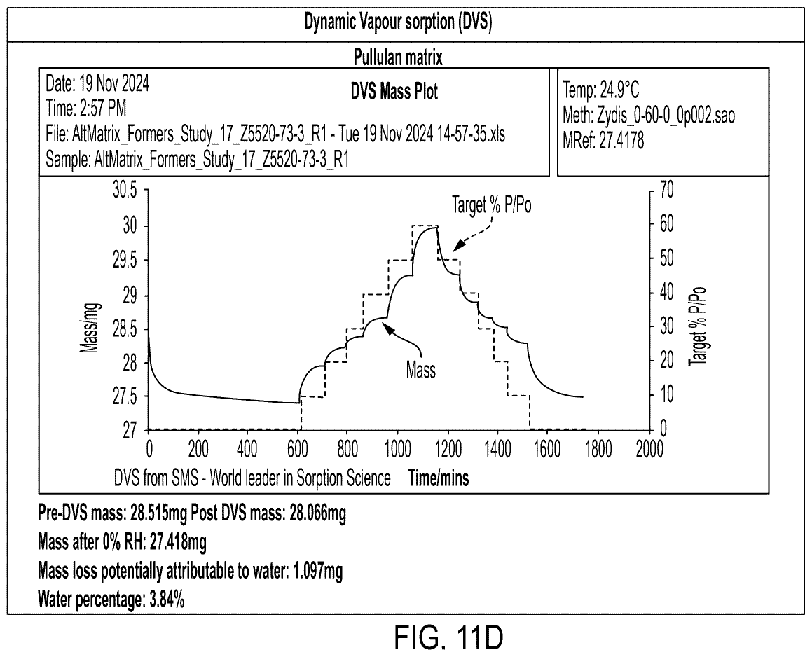

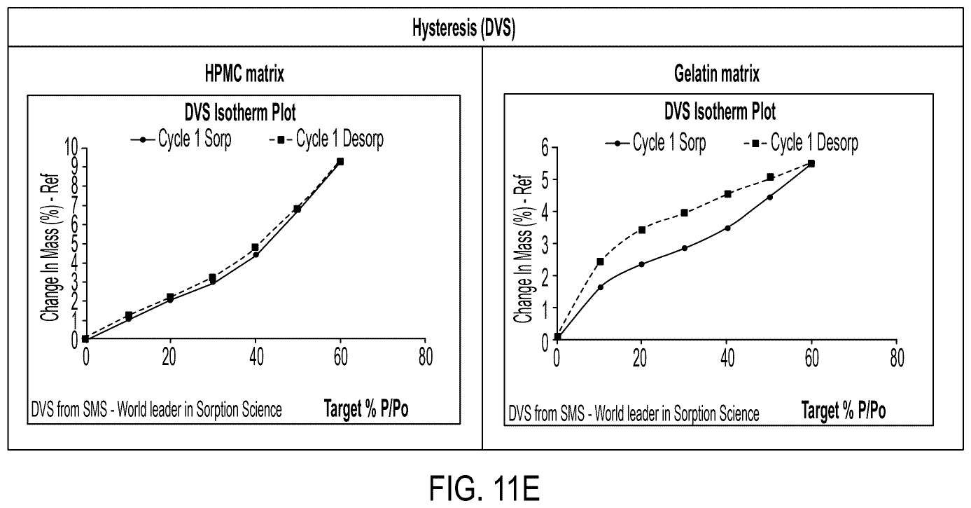

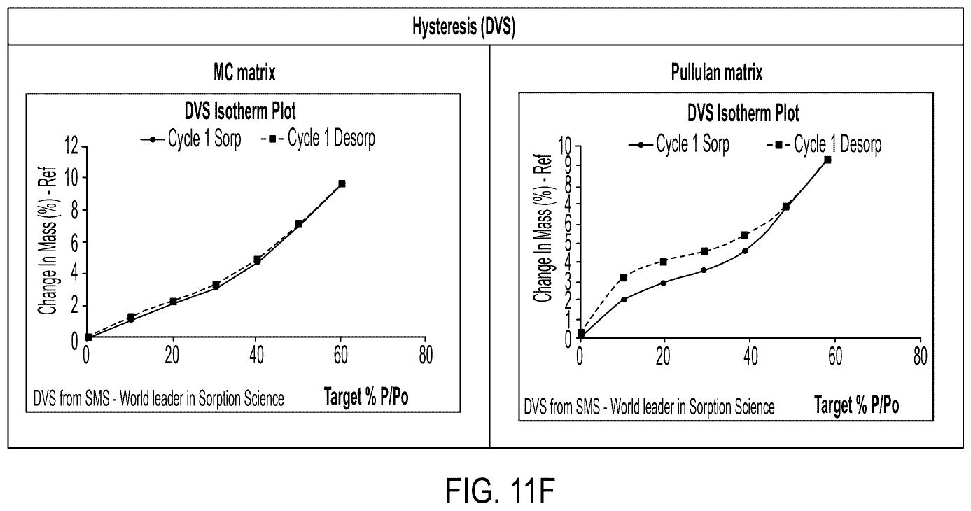

FIGS. 11A-F illustrate dynamic vapour sorption (DVS) and hysteresis results for the pharmaceutical compositions used in the studies described herein. For DVS the following settings were used: Range 0-60% with 10% steps (increments) at a dm/dt (change in mass/change in time) of 0.002% per minute and a temperature of 25° C. Dynamic Vapor Sorption (DVS) analysis demonstrated that non-gelatin matrix formers, including hydroxypropyl methylcellulose (HPMC) and methylcellulose (MC), exhibit faster moisture sorption kinetics compared to gelatin-based systems. In contrast, the gelatin matrix units displayed a more gradual moisture uptake profile, attributed to structural transformations such as swelling or gel formation during exposure to humidity. Post-sorption mass measurements indicated a net mass decrease for HPMC, MC, and Pullulan matrix formers, whereas the gelatin matrix former unit exhibited a net mass gain, consistent with the retention of bound water. Furthermore, the relatively narrow hysteresis loops observed for HPMC and MC suggest moderate porosity with reversible moisture sorption characteristics, which are advantageous for enhancing formulation stability. As such, HPMC and MC may be less susceptible to retain water than gelatin

In some embodiments, the non-gelatin matrix former exhibits a narrow hysteresis loop during dynamic vapor sorption analysis, indicating moderate porosity and reversible moisture uptake. In some embodiments, the pharmaceutical composition's (with a moisture sensitive API) stability can be enhanced by embedding the API in a non-gelatin matrix former, wherein the matrix provides reversible moisture sorption characteristics and reduced bound water retention compared to gelatin matrix formers. In some embodiments, the pharmaceutical composition maintains physical integrity and stability under fluctuating humidity conditions due to the reversible moisture sorption behavior of the matrix.

### Buffering Capacity Studies

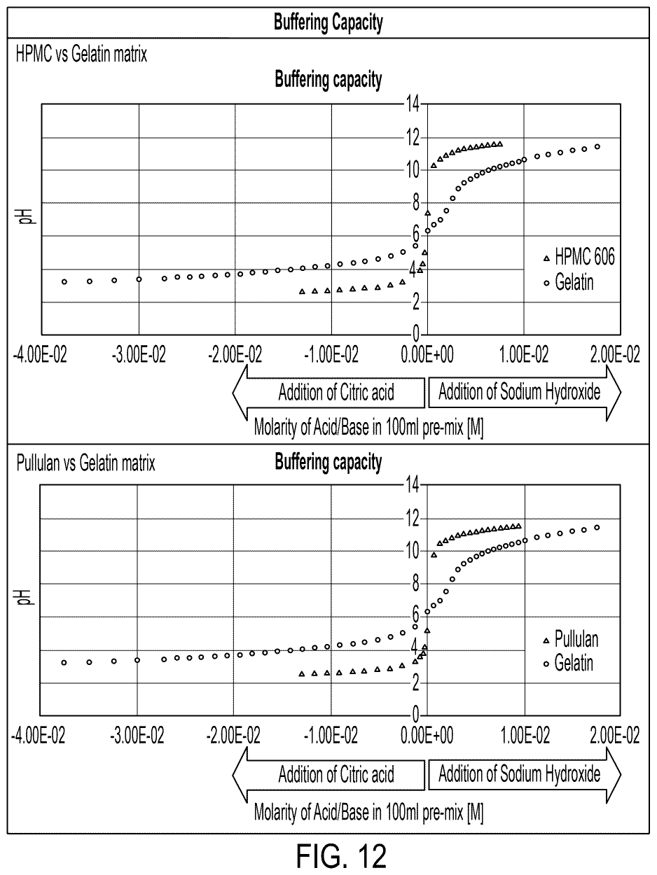

FIG. 12 illustrates buffering capacity results in the buffering capacity study. For buffering capacity, two solutions were prepared per matrix former (1. HPMC 4%+Mannitol 5%+Purified water (qs 100%); 2. Pullulan 5%+Mannitol 3%+Purified water (qs 100%)). To one solution, 10% NaOH was added in specified increments and the pH recorded. To the second solution, 10% citric acid sol was added in specified increments and the pH recorded. The buffering capacity study demonstrates that formulations utilizing hydroxypropyl methylcellulose (HPMC) and pullulan as matrix-forming agents exhibit selective buffering behavior. Buffering capacity is limited between ˜ pH 4 and 10, as evidenced by rapid pH shifts upon addition of citric acid or sodium hydroxide. However, outside this range, the matrix formers show enhanced pH stability, indicating saturation or stronger resistance to further pH modification.

Importantly, gelatin-based matrix formers are known to degrade under highly acidic (pH<4) and highly alkaline (pH>10) conditions, leading to compromised mechanical integrity and potential loss of drug containment. In contrast, HPMC and pullulan matrix formers maintain structural stability across a broader pH range, making them more suitable for the delivery of drugs that require formulation in extreme pH environments. This property, combined with the reduced need for hygroscopic pH modifiers such as citric acid and sodium hydroxide, can contribute to improved moisture resistance, shelf life, and/or overall robustness of the final product.

In some embodiments, the non-gelatin matrix former exhibits selective buffering behavior with limited buffering capacity between approximately a pH 4 and a pH 10, and enhanced pH stability outside this range. In some embodiments, the non-gelatin matrix former resists rapid pH changes upon addition of acidic or alkaline agents outside the pH range of 4 to 10. In some embodiments, the non-gelatin matrix former maintains structural integrity under highly acidic (pH<4) and highly alkaline (pH>10) conditions. In some embodiments, the non-gelatin matrix former eliminates or reduces the need for hygroscopic pH modifiers such as citric acid or sodium hydroxide. In some embodiments, the non-gelatin matrix former can be used when the pharmaceutical composition is intended for delivery in extreme pH environments such that the non-gelatin matrix former maintains mechanical integrity and drug containment across a pH range of less than 4 to greater than 10. In some embodiments, the non-gelatin matrix former provides improved moisture resistance and extended shelf life due to reduced reliance on hygroscopic pH modifiers. In some embodiments, the non-gelatin matrix former can be selected to avoid degradation and maintain robustness under extreme pH conditions, unlike gelatin-based matrices.

### Additional Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “about X” includes description of “X”. In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters. For example, a statement that a solution has a wt. % of at least about 10 wt. %, about 15 wt. %, or about 20 wt. % is meant to mean that the solution has a weight percent of at least about 10 wt. %, at least about 15 wt. %, or at least about 20 wt. %.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown but is to be accorded the widest scope consistent with the principles and features disclosed herein.

1. A pharmaceutical composition, comprising:
an active pharmaceutical ingredient (“API”);
1-45 wt. % non-gelatin matrix former comprising hydroxypropyl methylcellulose, methyl cellulose, pullulan, or combinations thereof; and
1-45 wt. % structure former.

2. The pharmaceutical composition of claim 1, wherein the pharmaceutical composition comprises 1-95 wt. % API.

3. The pharmaceutical composition of claim 1, wherein the structure former comprises sugars, polyols, or combinations thereof.

4. The pharmaceutical composition of claim 1, wherein the structure former comprises isomalt, sorbitol, mannitol, sucralose, trehalose, dextrose, lactose, galactose, cyclodextrin, or combinations thereof.

5. The pharmaceutical composition of claim 4, wherein the structure former comprises isomalt, sorbitol, mannitol, sucralose, trehalose, or combinations thereof.

6. The pharmaceutical composition of claim 1, further comprising 0.001-2 wt. % pH modifier and 0.001-1 wt. % flavoring agent.

7. The pharmaceutical composition of claim 1, wherein a disintegration time of the pharmaceutical composition is less than 7 seconds.

8. The pharmaceutical composition of claim 1, wherein the pharmaceutical composition comprises at most 5 wt. % water.

9. The pharmaceutical composition of claim 1, wherein the pharmaceutical composition is an orally dissolvable tablet.

10. The pharmaceutical composition of claim 1, wherein a percent dissolution of the pharmaceutical composition and corresponding API after 6 minutes is at least 75% of label claim.

11. The pharmaceutical composition of claim 1, wherein a water activity of the pharmaceutical composition is 0.2-0.45.

12. The pharmaceutical composition of claim 1, wherein the non-gelatin matrix former exhibits a narrow hysteresis loop during dynamic vapor sorption analysis, indicating moderate porosity and reversible moisture uptake.

13. The pharmaceutical composition of claim 1, wherein the API is embedded in the non-gelatin matrix former such that the non-gelatin matrix former provides reversible moisture sorption characteristics and reduced bound water retention compared to a gelatin matrix former.

14. The pharmaceutical composition of claim 13, wherein the pharmaceutical composition maintains physical integrity and stability under fluctuating humidity conditions due to the reversible moisture sorption behavior of the non-gelatin matrix former.

15. The pharmaceutical composition of claim 14, wherein the non-gelatin matrix former exhibits selective buffering behavior with limited buffering capacity between a pH of 4-10.

16. The pharmaceutical composition of claim 1, wherein the non-gelatin matrix former resists rapid pH changes upon addition of acidic or alkaline agents outside the pH range of 4-10.

17. A method of making a pharmaceutical composition, the method comprising:
dosing a pharmaceutical formulation into a preformed mold, the pharmaceutical formulation comprising:
an active pharmaceutical ingredient (“API”);
1-20 wt. % non-gelatin matrix former comprising hydroxypropyl methylcellulose, methyl cellulose, pullulan, or combinations thereof; and
1-20 wt, % structure former;
freezing the dosed pharmaceutical formulation; and
freeze-drying the frozen pharmaceutical formulation to form the pharmaceutical composition.

18. The method of claim 17, wherein the dosed pharmaceutical formulation is frozen at a temperature of −40° C. to −120° C. for a duration of about 1-5 minutes.

19. The method of claim 17, wherein the pharmaceutical formulation comprises 1-45 wt. % API.

20. The method of claim 17, wherein the pharmaceutical formulation further comprises 0.001-5 wt. % pH modifier and 0.001-5 wt. % flavoring agent.

21. A pharmaceutical composition, comprising:
25-35 wt. % an active pharmaceutical ingredient (“API”);
20-45 wt. % non-gelatin matrix former comprising hydroxypropyl methylcellulose, methyl cellulose, pullulan, or combinations thereof; and
20-45 wt. % structure former comprising isomalt, mannitol, sorbitol, sucralose, trehalose, or combinations thereof.

22. The pharmaceutical composition of claim 21, wherein the API is loratadine.

23. A pharmaceutical composition, comprising:
40-60 wt. % an active pharmaceutical ingredient (“API”);
20-35 wt. % non-gelatin matrix former comprising hydroxypropyl methylcellulose, methyl cellulose, pullulan, or combinations thereof; and
15-35 wt. % structure former comprising isomalt, mannitol, sorbitol, sucralose, trehalose or combinations thereof.

24. The pharmaceutical composition of claim 23, wherein the API is ibuprofen lysine.

25. A pharmaceutical composition, comprising:
85-95 wt. % an active pharmaceutical ingredient (“API”);
1-5 wt. % non-gelatin matrix former comprising hydroxypropyl methylcellulose, methyl cellulose, pullulan, or combinations thereof, and 1-5 wt. % structure former comprising isomalt, mannitol, sorbitol, sucralose, trehalose, or combinations thereof.

26. The pharmaceutical composition of claim 25, wherein the API is ibuprofen.
